(19) United States
(12) Patent Application Publication
(10) Pub. No.: US 2024/0254218 A1
(43) Pub. Date: Aug. 1, 2024

(54) COMPOSITIONS AND METHODS FOR TREATING NEURODEGENERATIVE DISEASES BY INHIBITING FSH

(71) Applicants: ICAHN SCHOOL OF MEDICINE AT MOUNT SINAI, New York, NY (US); EMORY UNIVERSITY, Atlanta, GA (US)

(72) Inventors: Mone Zaidi, New York, NY (US); Keqiang Ye, Atlanta, GA (US)

(21) Appl. No.: 18/290,633

(22) PCT Filed: Jul. 21, 2022
(86) PCT No.: PCT/US2022/037882

§ 371 Date: Jan. 19, 2024

Related U.S. Application Data
(60) Provisional application No. 63/224,092, filed on Jul. 21, 2021.

(51) Int. Cl. C07K 16/26; A61K 39/00; A61K 45/06; A61P 25/28

(52) U.S. Cl. CPC C07K 16/26; A61K 45/06; A61P 25/28; A61K 2039/505; C07K 2317/34; C07K 2317/76

(57) ABSTRACT

The present disclosure provides compositions and methods for treating neurodegenerative diseases, in particular, Alzheimer's Disease, by inhibiting FSH in a subject in need thereof. A method comprising of treating Alzheimer's Disease (AD), preventing the onset of AD, or reducing cognitive or functional decline in AD, in a subject in need or at risk thereof.

### CROSS-REFERENCE TO RELATED APPLICATIONS

This application claims priority to U.S. Provisional Application No. 63/224,092, filed Jul. 21, 2021. The contents of this application are incorporated herein by reference in its entirety.

### STATEMENT REGARDING FEDERAL FUNDING

This invention was made with government support of U19 AG60917, U19 AG60917-02S1, AG065177 and AG051538 awarded by the National Institutes of Health/National Institute on Aging. The government has certain rights in the invention.

### TECHNICAL FIELD

This disclosure relates generally to methods of treating neurodegenerative diseases in a subject by inhibiting FSH. In particular, the methods of the disclosure can be used to treat Alzheimer's Disease (AD).

### BACKGROUND

Neurodegenerative diseases of the aging population such as Alzheimer's Disease (AD) affect about 35 million people worldwide, with an estimated 7 million new cases every year (World Health Organization; Dementia Fact Sheet; September 2020). AD and other neurodegenerative disorders pose a major global health problem resulting in progressive dementia, profound disability, and impaired quality of life. Constituting ˜70% of the AD population (Andersen, K. et al. Neurology 53, 1992-1997, (1999)), women have a greater life-time risk for AD than men, and display a ˜3-fold higher rate of disease progression (Laws, K. R., et al. World J Psychiatry 6, 54-65, (2016)) with a broader spectrum of behavioral symptoms (Koran, M. E. I., et al. Brain Imaging Behav 11, 205-213, (2017); Marongiu, R. Front Aging Neurosci 11, 242, (2019)).

Yet, there are few treatment options for Alzheimer's Disease and other neurodegenerative diseases regardless of gender, with current medications unable to stop the damage to neurons, and limited symptomatic-only treatments available. Thus, there is an urgent and wide-ranging need for treatments of neurodegenerative diseases, such as Alzheimer's Disease, which are associated with beta-amyloid deposits and/or neurofibrillary tangles.

### SUMMARY

The present disclosure is based, at least in part, on the unexpected discovery that follicle-stimulating hormone (FSH) acts on hippocampal neurons to accelerate amyloid β and Tau deposition and impairs cognitive function in a mouse model of Alzheimer's Disease (AD), and that anti-FSH antibodies can attenuate cognitive decline and AD neuropathology.

Disclosed herein is a method of treating Alzheimer's Disease (AD), preventing the onset of AD, or reducing cognitive or functional decline in AD, in a subject in need or at risk thereof, comprising administering to said subject a therapeutically effective amount of a composition comprising an FSH-inactivating agent.

In some embodiments, the subject is female. In some embodiments, the subject is perimenopausal or postmenopausal. In some embodiments, the subject is male. In some embodiments, the subject is perimenopausal or postmenopausal.

In some embodiments, the subject has a condition in which FSH levels are elevated. In some embodiments, the condition is a genetic disease, chemotherapy, surgical menopause, or orchiectomy.

In some embodiments, the genetic disease is Turners syndrome.

In some embodiments, the method alters one or more of the following in the subject in need thereof. (a) reduces Aβ accumulation; (b) reduces amyloid plaques; (c) reduces Tau accumulation in the brain; and (d) enhances cognitive function.

In some embodiments, the one or more of Aβ accumulation, amyloid plaques, and Tau accumulation in the brain is lower by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the subject or in a control.

In some embodiments, the cognitive function is enhanced by at least about 20%, at least about 30%, at least about 40%, or at least about 50%, at least about 60%, at least about 70%, at least about 80%, or at least about 90%, as measured on one or more tests selected from the group consisting of the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test; the Verbal Paired Associates subtest; the Logical Memory subtest: the Visual Reproduction subtest of the Wechsler Memory Scale-Revised (WMS-R); the explicit 3—alternative forced choice task; and the Benton Visual Retention Test.

In some embodiments, the subject is concurrently treated with one or more agents selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, an adeno-associated virus delivery of NGF, CERE-110, a beta-blocker, a human amyloid vaccine, a beta or gamma secretase inhibitor, a nicotinic or muscarinic agonist, and a second antibody.

In some embodiments, the cholinesterase inhibitor is selected from the group consisting of galantamine, rivastigmine, tacrine, and donepezil. In some embodiments, the NMDA receptor antagonist is selected from the group consisting of ketamine, methadone, memantine, amantadine, and dextromethorphan or a salt thereof. In some embodiments, the antipsychotic agent is selected from the group consisting of aripiprazole, risperidone, olanzapine, quetiapine, or haloperidol. In some embodiments, the benzodiazepine is selected from the group consisting of lorazepam, oxazepam and temazepam. In some embodiments, the tricyclic antidepressant is nortriptyline. In some embodiments, the hormone is selected from the group consisting of estrogen, progesterone and leuprolide. In some embodiments, the vitamin selected from the group consisting of folate and nicotinamide.

In some embodiments, the second antibody is selected from the group consisting of bapineuzumab, solanezumab, gantenerumab, crenezumab, ponezumab, BAN2401, and aducanumab.

In some embodiments, the FSH-inactivating agent is administered intravenously, intrathecally, intracranially, cutaneously, subcutaneously, intraperitoneally, intramuscularly, orally, topically, transdermally, nasally, or rectally to the subject.

In some embodiments, the FSH-inactivating agent comprises an anti-FSH antibody, or antigen-binding portion thereof, wherein the anti-FSH antibody, or antigen-binding portion thereof specifically binds to one or more epitopes within a β-subunit of Follicle stimulating hormone (FSH).

In some embodiments, the one or more epitopes within the β-subunit of FSH comprise SEQ ID NO: 1 or SEQ ID NO: 2 or a peptide sequence consisting essentially of SEQ ID NO: 1 or SEQ ID NO: 2 but having conservative substitutions.

In some embodiments, the anti-FSH antibody or antigen-binding portion thereof is selected from the group consisting of a monoclonal antibody, a polyclonal antibody, a chimeric antibody, a human antibody, a single-chain variable fragment (scFv), and combinations thereof. In some embodiments, the anti-FSH antibody is a polyclonal antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows expression of Aβ and Tau (AT8) determined by immunohistochemistry in the hippocampus of sham or ovariectomized (OVX) female mice, injected with goat IgG (control; first two columns) or FSH Ab (last two columns). Silver staining shows proteinaceous deposits in the tissue. Female 3xTg mice underwent ovariectomy (OVX) or sham operation (Sham), and were injected with anti-FSHβ (FSH Ab) or goat IgG (200 μg/mouse, i.p., every two days) for 8 weeks. Scale bar, 50 μm.

FIG. 1B shows fold change in the expression of Aβ40 and Aβ42 as measured by ELISA in sham or ovariectomized (OVX) female mice. The columns on the graphs are control (IgG) vs FSH Ab (Ab) from left to right per group. (N=3 biological replicates/group, one-way ANOVA, *P<0.05, **P<0.01.

FIG. 1C shows western immunoblots for expression of C/EBPβ, AEP, cleaved APP, Tau and phosphorylated Tau protein in the brains of sham or ovariectomized (OVX) female mice injected with IgG or FSH Ab. Control: β-actin.

FIG. 1D shows asparagine endopeptidase (AEP/S-secretase) activity in brain extracts of sham or ovariectomized (OVX) female mice, injected with goat IgG (left column in each group) or FSH Ab (right column in each group). Mean±SEM, N=3, one-way AVOVA, *P<0.05, **P<0.01.

FIG. 1E show graphs depicting latency to platform (seconds) (left graph); integrated escape latency (area under the curve, AUC; middle graph) and percentage of time spent in the target quadrant (Probe Trial Test; right graph) as measured during Morris water maze testing over five training days in sham or ovariectomized (OVX) female mice, injected with goat IgG (IgG) or FSH Ab (Ab). N=9 mice/group, one-way ANOVA, *P<0.05, **P<0.01.

FIG. 1F shows hippocampal and cortical Aβ40 and Aβ42 accumulation (pg/pg measured by ELISA) in wild-type (WT) or Amyloid Beta Precursor Protein/Presenilin-1 (APP/PS1) transgenic male mice treated with goat IgG (left bar in each group) or FSH Ab (right bar in each group). Statistics: mean±SEM, two-tailed Student's t-test, N=4 to 10 mice/group, *P<0.05; *P<0.01 or as shown. Mice were genotyped and re-genotyped for human PSEN1, followed by qPCR for human APP.

FIG. 2A shows relative expression of Follicle-stimulating hormone receptor (Fshrs) in the human cortex, human neuroblastoma cells (SH-SY5Y), mouse cortex, mouse hippocampus, mouse ovary and rat cortical neurons as determined by polymerase chain reaction (PCR).

FIG. 2B shows expression of Follicle-stimulating hormone receptor (Fshrs) in the mouse cortex, hippocampus, hypothalamus, and ovary as determined by quantitative polymerase chain reaction (PCR).

FIG. 2C shows relative expression of Follicle-stimulating hormone receptor (Fshrs) in male and female whole mouse brains as determined by Western blotting.

FIG. 2D shows Western immunoblots of the expression levels of C/EBPβ, Tau, Tau¹⁻³⁶⁸, AEP, APP, APP¹⁻⁵⁸⁵ and FSHR in control SH-SY5Y cells, cells with reduced expression of Cebpb using short hairpin (sh)RNA shRNA-Cebpb (shCebpb) or cells with reduced δ-secretase activity by adeno-associated virus infection of dominant-negative AEP^{C189S}. The cells were treated with PBS or FSH (30 ng/ml, 48 hour).

FIG. 2E shows Western immunoblots of the expression levels of C/EBPβ, AEP, pAEP^{S226}, total and phosphorylated AKT^{S473} (pAKT^{S473}), total and phosphorylated ERK1/2 (pERK1/2), total and phosphorylated SRPK2^{T492} (pSRPK2^{T492}) in SH-SY5Y cells incubated with FSH (30 ng/ml; 30 minutes) in the presence of DMSO (control), the cAMP inhibitor SQ22536 (100 μM), Gαᵢ inhibitor pertussis toxin (PTX, 50 ng/ml), AKTi-1/2 inhibitor (10 μM) and ERK1/2 inhibitor PD98059 (10 μM). kDa: kilodalton

FIG. 2F shows a schematic representation of the putative signaling cascade linking the FSHR with ERK1/2-AKT-SRPK2 phosphorylation, activation of C/EBPβ and AEP/δ-secretase, and APP and Tau cleavage and Tau phosphorylation.

FIG. 3A shows Western immunoblots of the expression levels of C/EBPβ, Tau, Tau¹⁻³⁶⁸, phosphorylated Tau (pTau), asparagine endopeptidase (AEP), cleaved amyloid precursor protein (APP), APP¹⁻⁵⁸⁵, APP¹⁻³⁷³ and FSHR in the whole brains of ovariectomized female 3xTg mice injected stereotactically with adeno-associated virus expressing either short hairpin Fshr RNA (iAAV04355302, shFshr) or control shRNA (iAAV01502; shControl).

FIG. 3B shows a graph depicting AEP activity (relative fluorescence units ×1000) in the brains of ovariectomized female 3xTg nice injected with sh-Control (left bar) or sh-Fshr (right bar). N=3 biological replicates; two-tailed Student's t-test, **P<0.01.

FIG. 3C shows immunofluorescence micrographs of NeuN neuronal marker (leftmost first column), Terminal deoxynucleotidyl transferase (TdT) dUTP Nick-End Labeling (TUNEL) to detect apoptotic cells (second column), 4′,6-diamidino-2-phenylindole (DAPI) staining to detect DNA (third column) and a merged overlay of NeuN, TUNEL and DAPI (last column) in OVX mice injected with sh-Control or shFshr. Scale bar, 20 μm.

FIG. 3D shows images of synapses (red arrows) in the brains of ovariectomized female 3xTg nice injected stereotactically with sh-Control (left panel) or sh-Fshr (right panel). The graph shows synapses per 100 μm² in sh-Control (left bar) or sh-Fshr (right bar); Scale bar, 1 μm. N=10 sections; two-tailed Student's t-test, *P<0.05.

FIG. 3E shows graphs depicting latency to platform (seconds) (left graph); integrated escape latency (area under the curve, AUC; middle graph) and percentage of time spent in the target quadrant (Probe Trial Test; right graph) as measured during Morris water maze testing over five training days in sham or ovariectomized (OVX) female mice, injected with sh-Control (AAV; left bar) or sh-Fshr (right bar). N=8 and 7 mice in shFshr and shControl groups, respectively; two-tailed Student's t-test, *P<0.05.

FIG. 4A shows Western immunoblots of expression levels of C/EBPβ, AEP/S-secretase, APP, cleaved APP¹³⁷³ and APP¹⁻⁵⁸⁵, total Tau, cleaved Tau¹³⁶⁸, phosphorylated Tau (pTau), FSHR, and 3-actin (control) in the brains of male (M) and female (F) 3xTg mice injected with PBS (control) or recombinant FSH (5 IU per mouse) daily, i.p. for 3 months.

FIG. 4B shows a graph depicting AEP/δ-secretase enzymatic activity (relative fluorescence units ×1000) in the brains of male (M) or female (F) 3xTg mice injected with PBS (control; left bar) or 5 IU/mouse recombinant FSH (right bar) daily, i.p. for 3 months. Mean±SEM, N=3, two-tailed Student's t-test, **P<0.01.

FIG. 4C show graphs depicting fold change percentage in the expression of Aβ isoforms Aβ40 and Aβ42 in the brains of male (M) or female (F) 3xTg mice injected with PBS (control; left bar) or 5 IU/mouse recombinant FSH (right bar) daily, i.p. for 3 months. Mean±SEM, N=3, two-tailed Student's t-test, **P<0.01.

FIG. 4D shows graphs depicting latency to platform (seconds) (top graph); integrated escape latency (area under the curve, AUC; lower left graph) and percentage of time spent in the target quadrant (Probe Trial Test; lower right graph) as measured during Morris water maze testing over five training days in male (M) or female (F) 3xTg mice injected with PBS (control; left bar) or 5 IU/mouse recombinant FSH (right bar). N=7 mice/group, two-tailed Student's t-test, *P<0.05, **P<0.01.

FIG. 4E shows electrophysiology for assessing long-term potentiation (LTP) in response to FSH. Traces are representative field excitatory post-synaptic potentials (fEPSPs) recorded before (black) and 60 min after (red) Tris-buffered saline (TBS). Mean±SEM, N=3, **P<0.01, two-way ANOVA and Bonferroni's posthoc test.

FIG. 5A shows Western immunoblots of levels of total C/EBPβ, AEP, cleaved APP, cleaved Tau, pTau, and FSHR in female 3xTg or 3xTg; Cebpb^{+/-} mice following PBS or FSH (5 IU/mouse, daily, i.p.) treatment for 3 months. kDa: kilodalton

FIG. 5B shows Western immunoblots of levels of total C/EBPβ, AEP, cleaved APP, cleaved Tau, pTau, and FSHR in female 3xTg or 3xTg;Cebpb^{+/-} mice in sham-operated or ovariectomized mice. kDa: kilodalton

FIG. 5C shows a graph depicting AEP enzymatic activity (relative fluorescence units×1000) in the brains of 3xTg mice (left column) or 3xTg;Cebpb^{+/-} mice (right column) injected with PBS or 5 IU/mouse recombinant FSH daily, i.p. for 3 months. Mean±SEM, N=3 animals per group, two-tailed Student's t-test, **P<0.01. N=4 biological replicates.

FIG. 5D shows a graph depicting AEP enzymatic activity (relative fluorescence units×1000) in the brains of 3xTg mice (sham-operated; left column); 3xTg mice (ovariectomized; middle column) or 3xTg;Cebpb^{+/-} mice (ovariectomized; right column). Mean±SEM, N=3 per group, N=4 biological replicates. One-way ANOVA, **P<0.01.

FIG. 5E shows graphs depicting latency to platform (seconds) (top graph); integrated escape latency (area under the curve, AUC; bottom left graph) and percentage of time spent in the target quadrant (Probe Trial Test; bottom right graph) as measured during Morris water maze testing over five training days in 3xTg mice (left column) or 3xTg;Cebpb^{+/-} mice (right column) following PBS or FSH treatment. N=9 animals/group. Two-tailed Student's t-test, *P<0.05.

FIG. 5F shows graphs depicting latency to platform (seconds) (top graph); integrated escape latency (area under the curve, AUC; bottom left graph) and percentage of time spent in the target quadrant (Probe Trial Test; bottom right graph) as measured during Morris water maze testing over five training days in 3xTg sham-operated mice (left column), 3xTg ovariectomized mice (middle column), or 3xTg;Cebpb^{+/-} ovariectomized mice (right column). N=7-8 animals/group. One-way ANOVA, *P<0.05, **P<0.01.

FIG. 6A shows images of the uterus in sham-operated and ovariectomized 3xTg mice. Note the hypoplastic thread-like uteri in ovariectomized 3xTg mice. The graphs show serum FSH levels (mIU/ml) and serum E₂ (pg/ml) in sham or ovariectomized mice administered control IgG (left column) or FSH Ab (right column; 100 μg/mouse/day, i.p., 3 months). Mean±SEM, N=8, one-way ANOVA, *P<0.05, **P<0.01.

FIG. 6B shows immunofluorescent micrographs with amyloid β (Aβ, red; first row in panels 1 and 2), thioflavin-s (green, second row in panel 1), APP^{C568} (green; second row in second panel), pTau (AT8; first row in panel 3) (red), Tau^{N368} (green; second row in panel 3), DAPI (third row in each panel), and merged images for panels 1-3 (fourth row in each panel) in the hippocampus of ovariectomized mice post treatment with control IgG (IgG) or FSH Ab (Ab). Scale bar, 20 μm.

FIG. 6C shows relative expression of Cebpb, Lgmn, App and Mapt in Sham and OVX mouse brains post treatment with control IgG (IgG; left column per group) or FSH Ab (Ab; right column per group). Mean±SEM, N=3, one-way AVOVA, *P<0.05, **P<0.01.

FIG. 6D shows immunofluorescence micrographs of NeuN neuronal marker (first row from top), Terminal deoxynucleotidyl transferase (TdT) dUTP Nick-End Labeling (TUNEL) to detect apoptotic cells (second row), 4′,6-diamidino-2-phenylindole (DAPI) staining to detect DNA (third row) and a merged overlay of NeuN, TUNEL and DAPI (last row) in Sham or OVX mice injected with control IgG (left column) or FSH Ab (right column). Scale bar, 20 μm.

FIG. 6E shows Golgi staining on brain sections from the CA1 hippocampal region, and the corresponding graph shows spine numbers (per 20 μm) in Sham or OVX animals treated with control IgG (left column) or FSH Ab (right column). Scale bar, 5 μm. Mean±SEM, N=8 sections, one-way ANOVA, *P<0.05, **P<0.01

FIG. 6F shows transmission electron micrographic images, and quantitative analysis of synapses in hippocampal sections from female 3xTg mice post-OVX treated with control IgG (left column) or FSH Ab (right column). (Scale bar, 1 m). Mean SEM, N=8 sections, one-way ANOVA, *P<0.05, **P<0.01.

FIG. 6G shows a graph depicting swim speed in Morris water maze testing in Sham, or OVX mice treated with control IgG (left column) or FSH Ab (right column). Statistics: N=9 mice/group, one-way ANOVA, *P<0.05, **P<0.01.

FIG. 611 shows binding (measured by absorbance at 450 nm) of FSH Ab or IgG to various concentrations of FSH or LH in an ELISA assay. Note the lack of cross-reactivity of FSH Ab with LH.

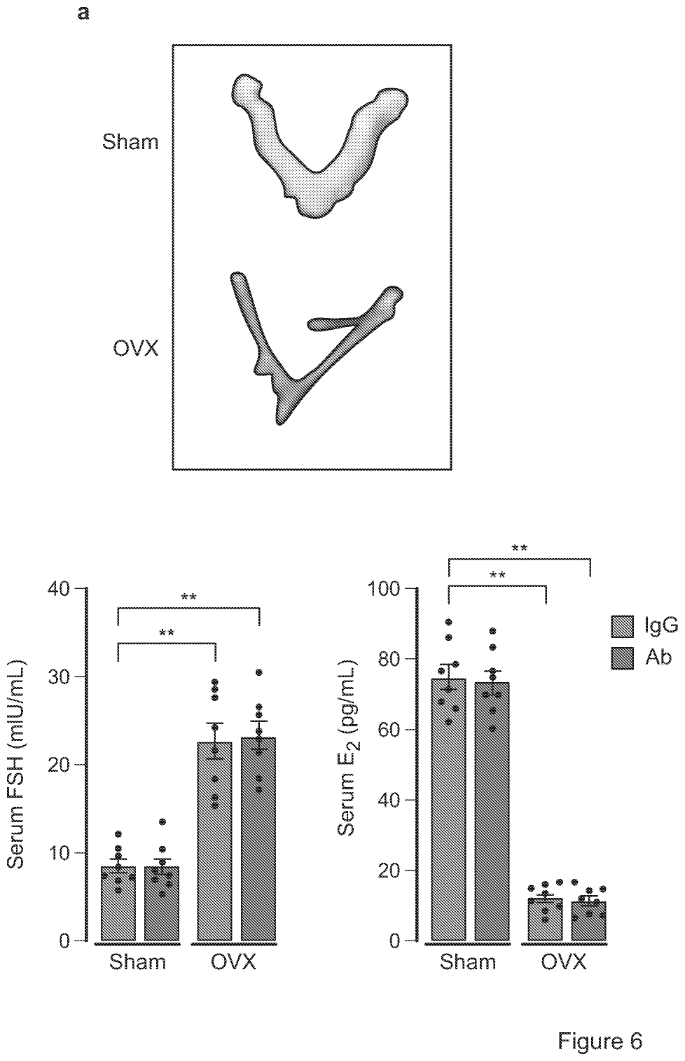

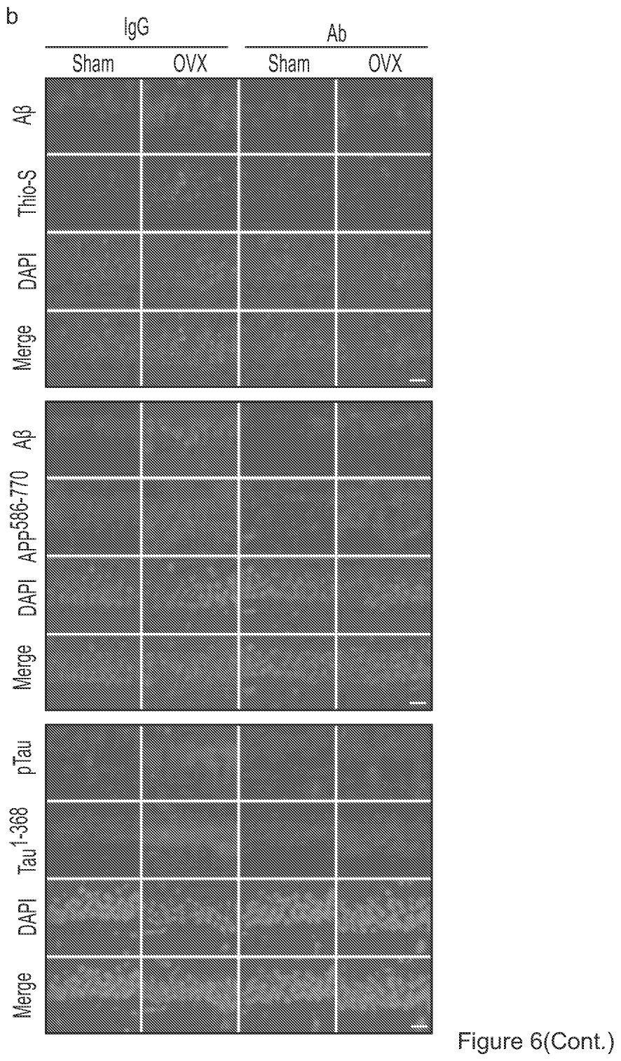

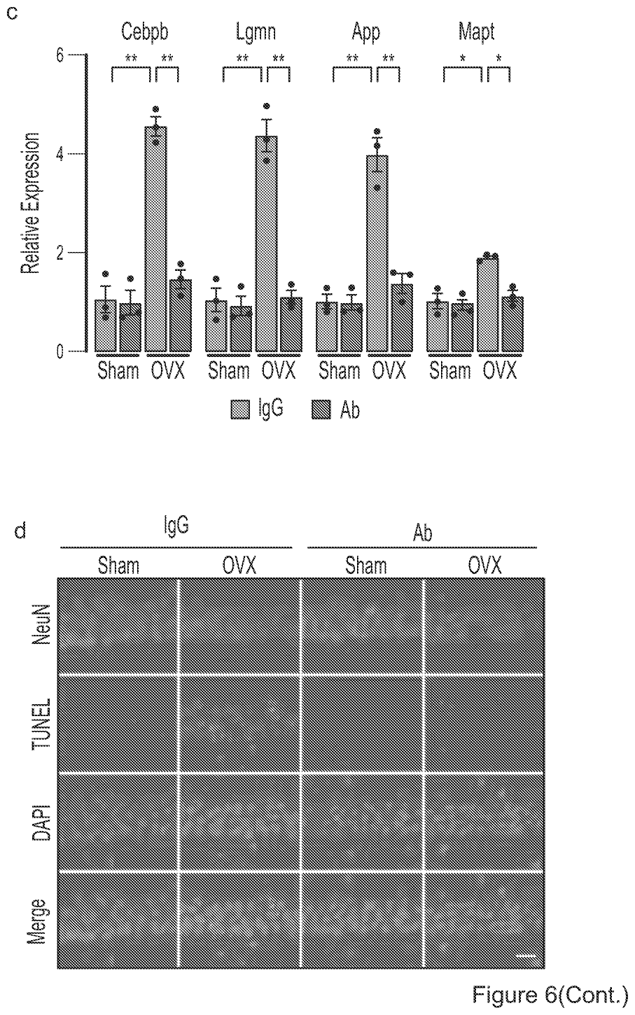

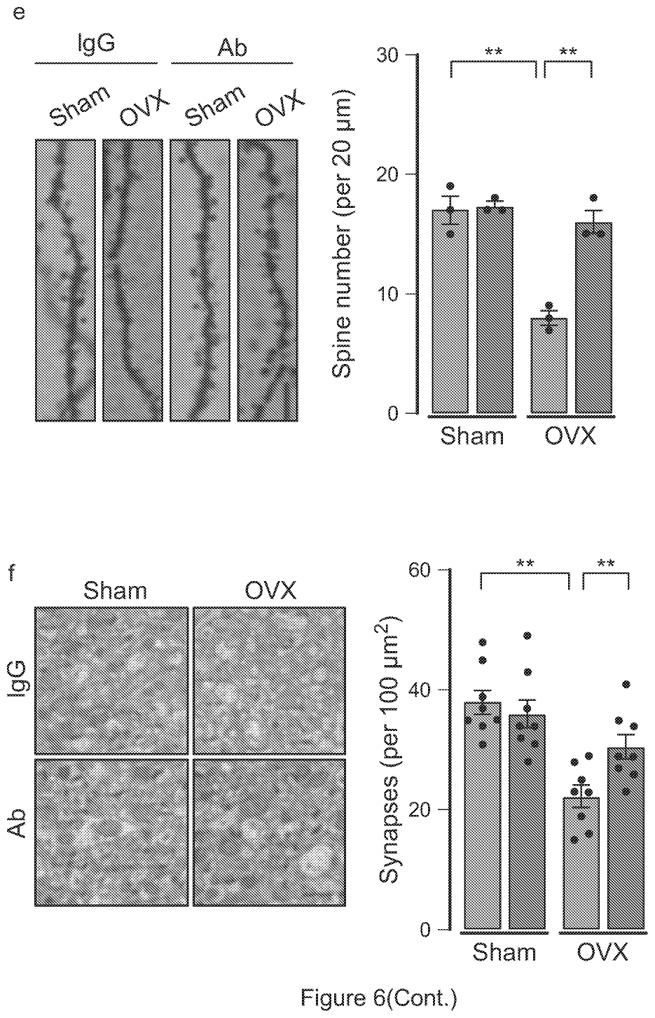

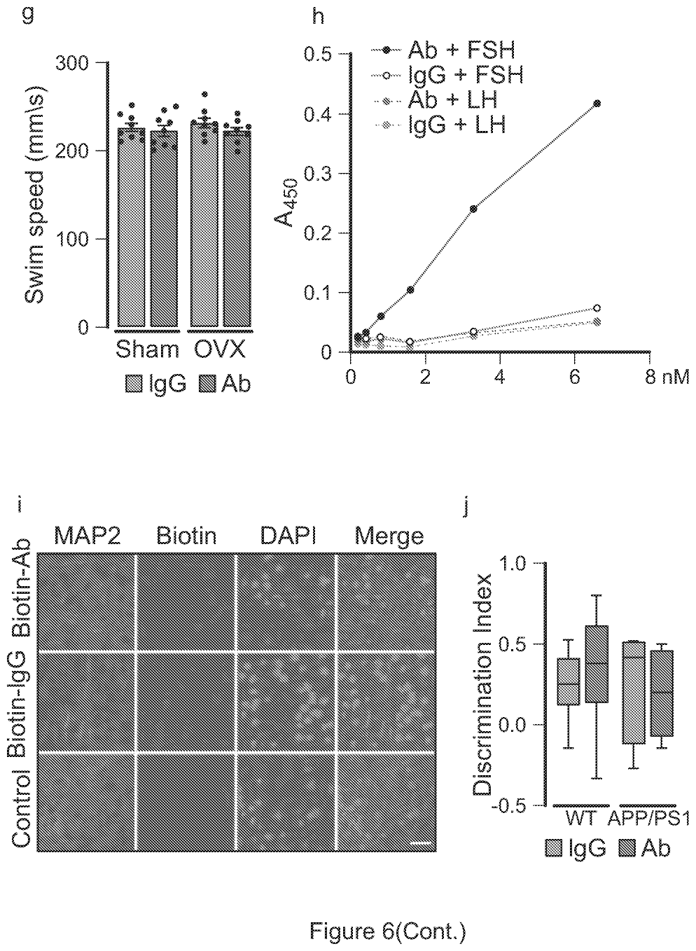

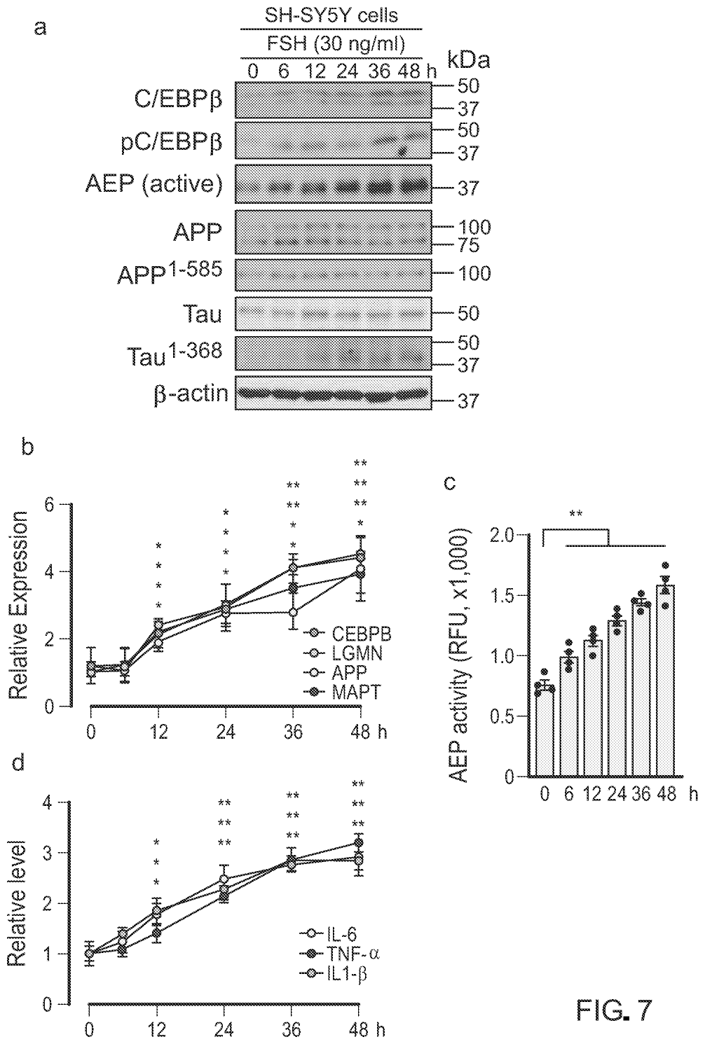

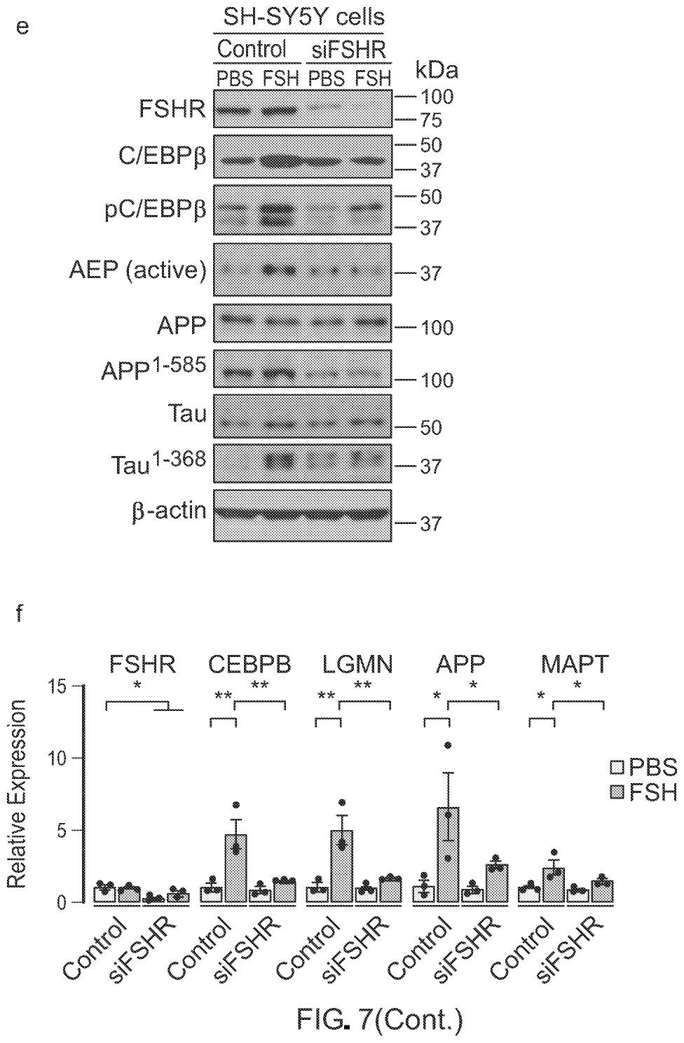

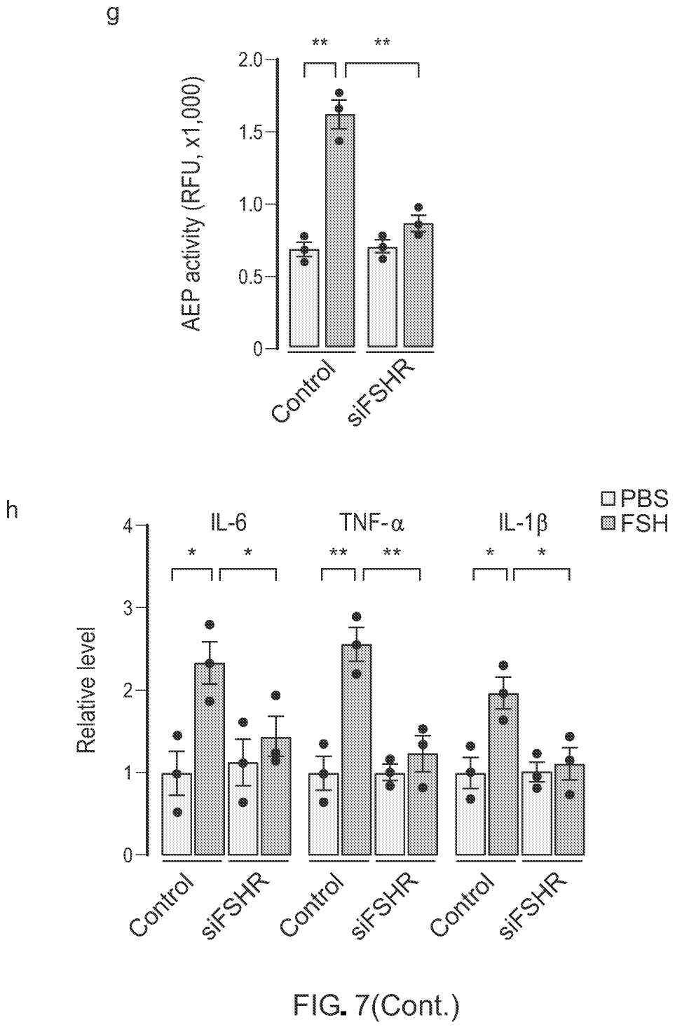

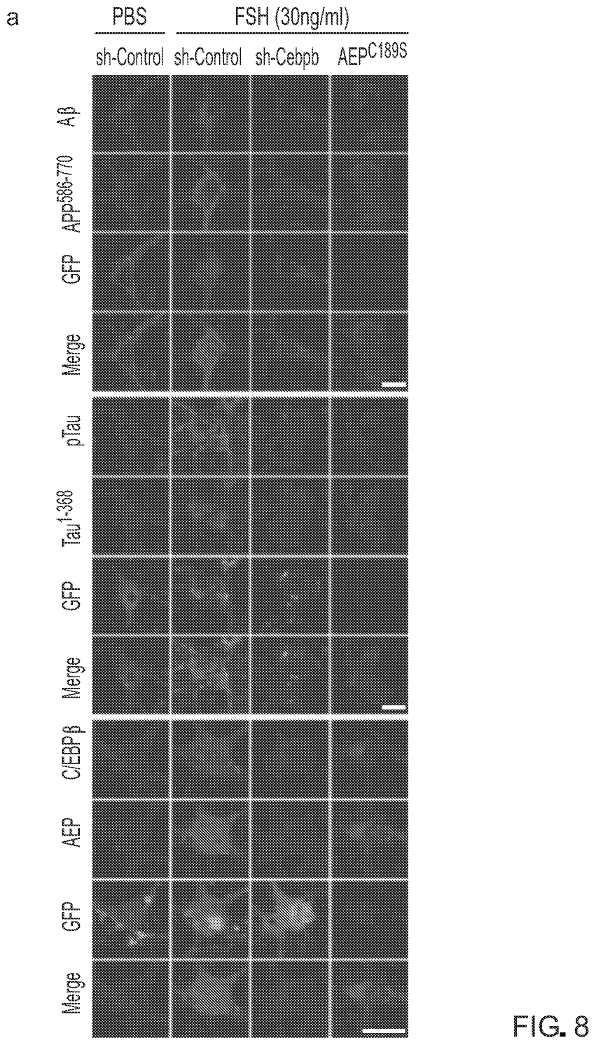

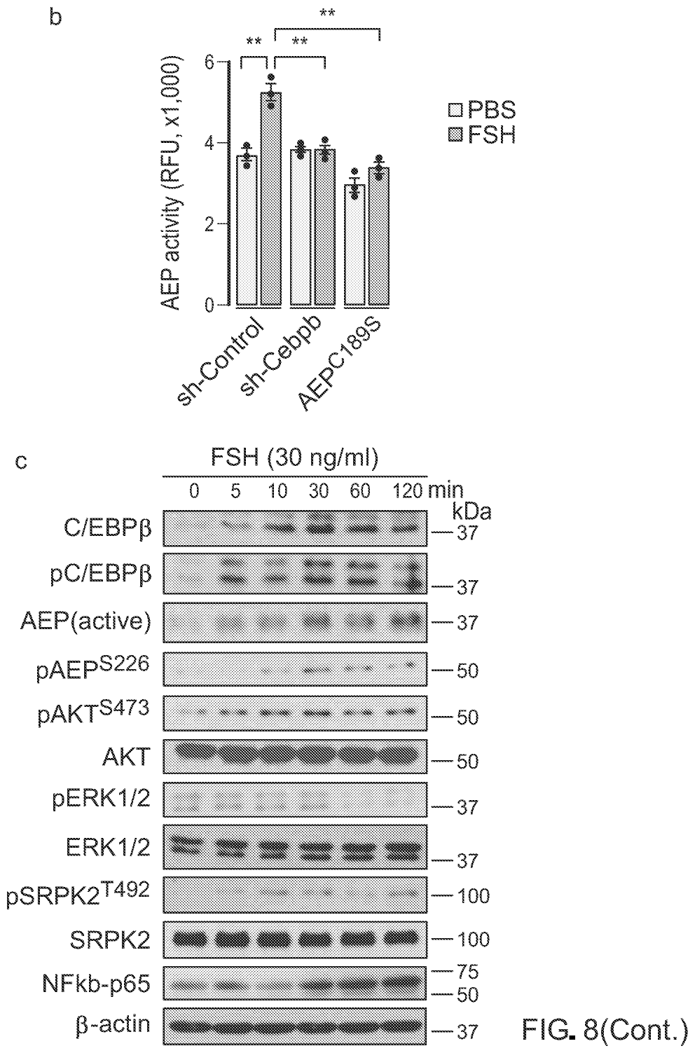

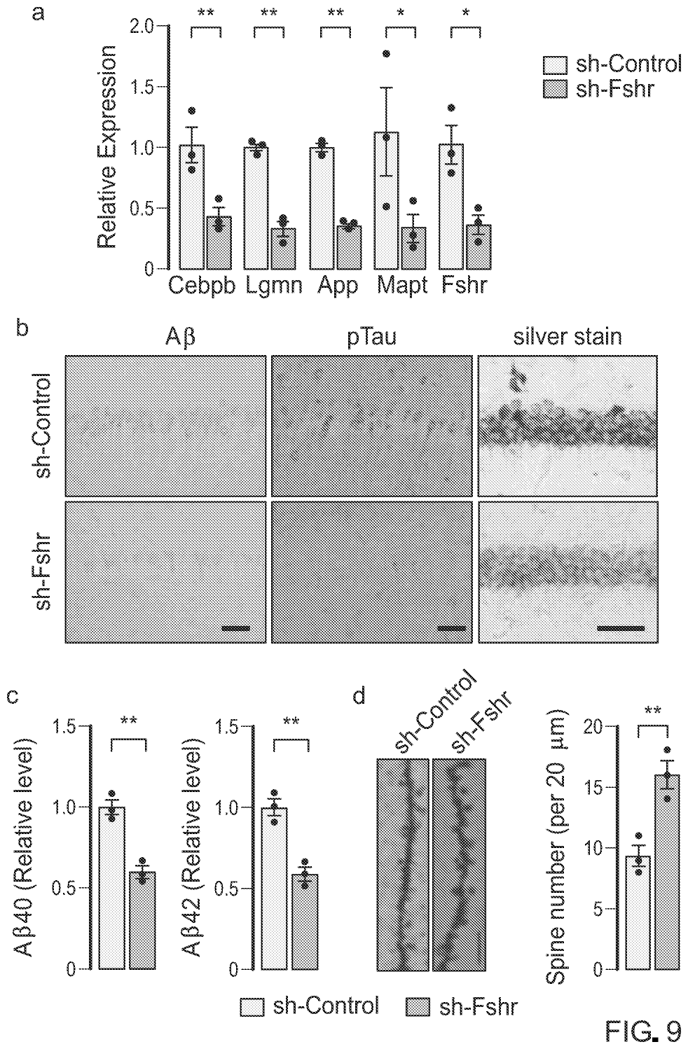

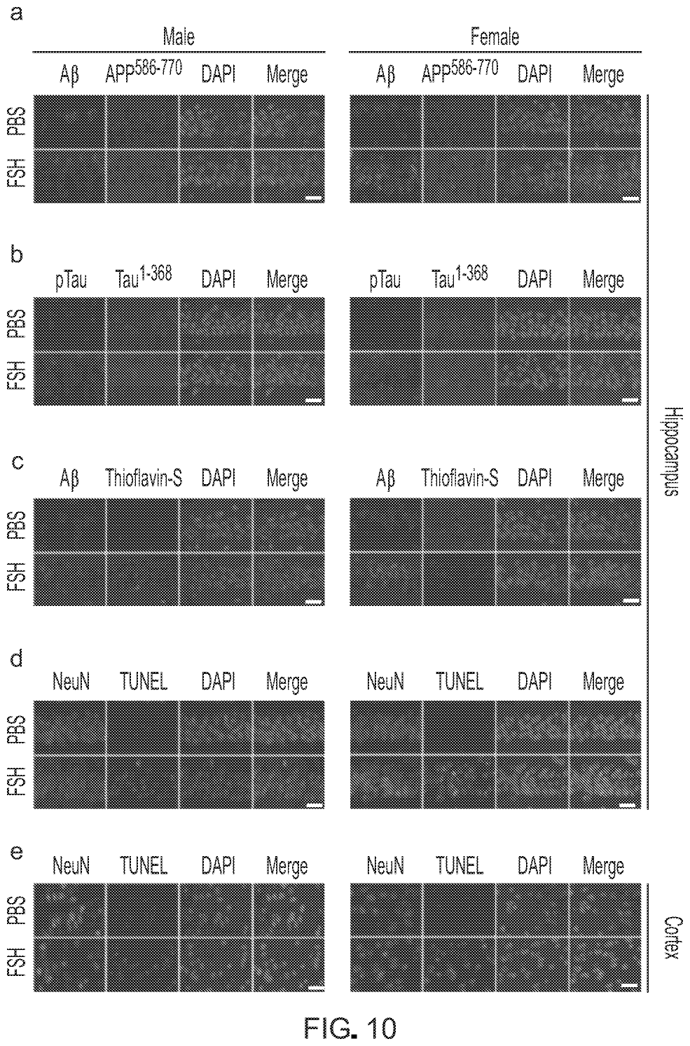

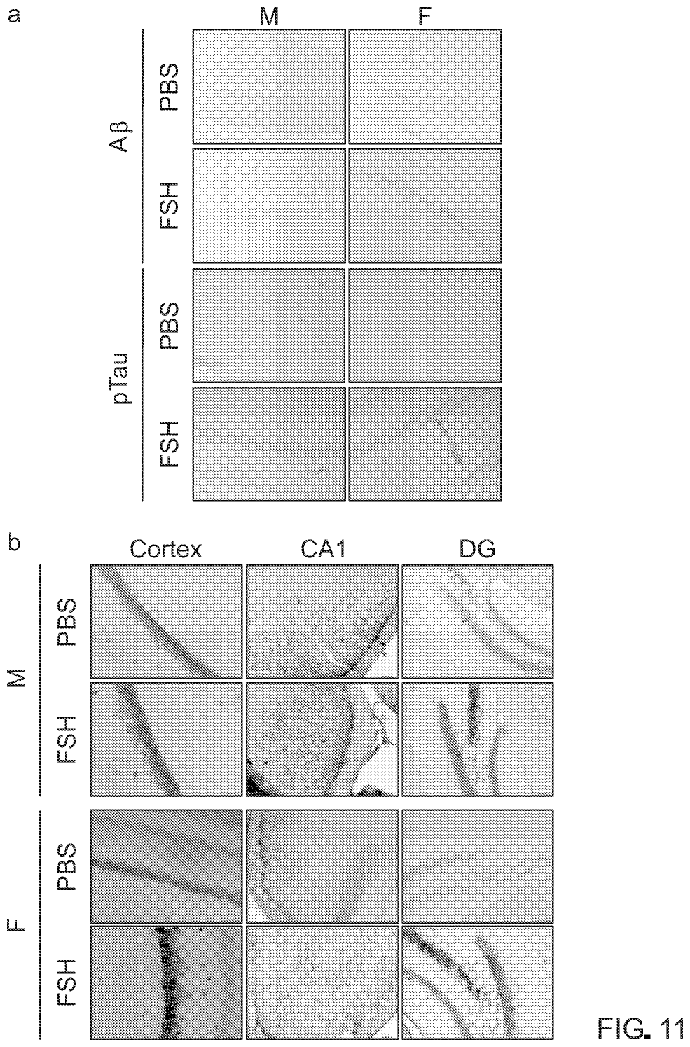

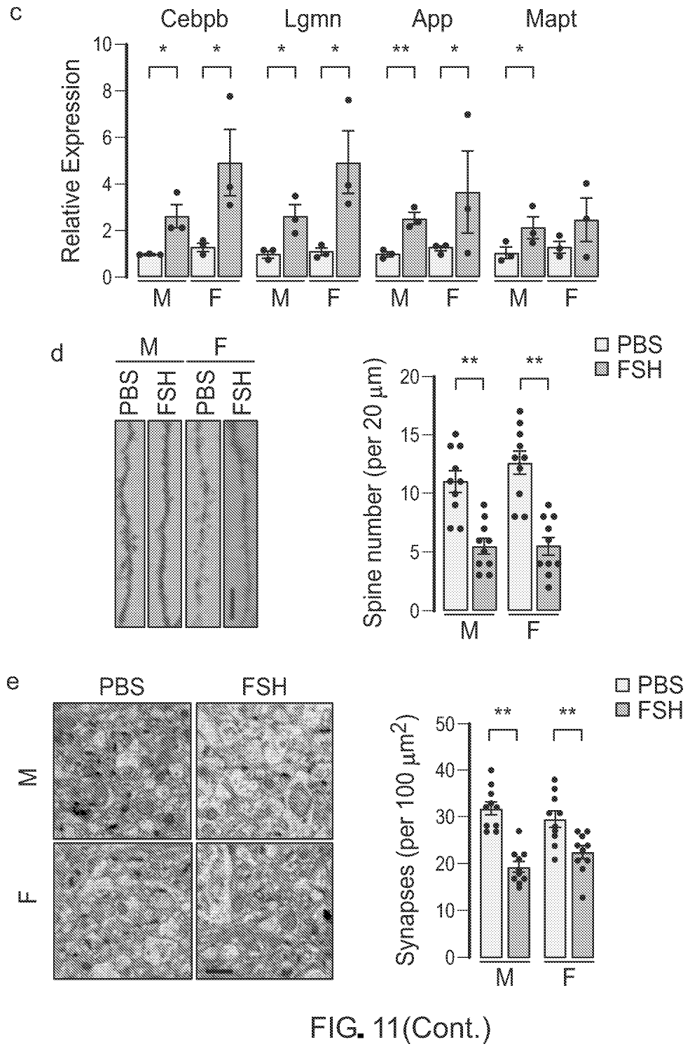

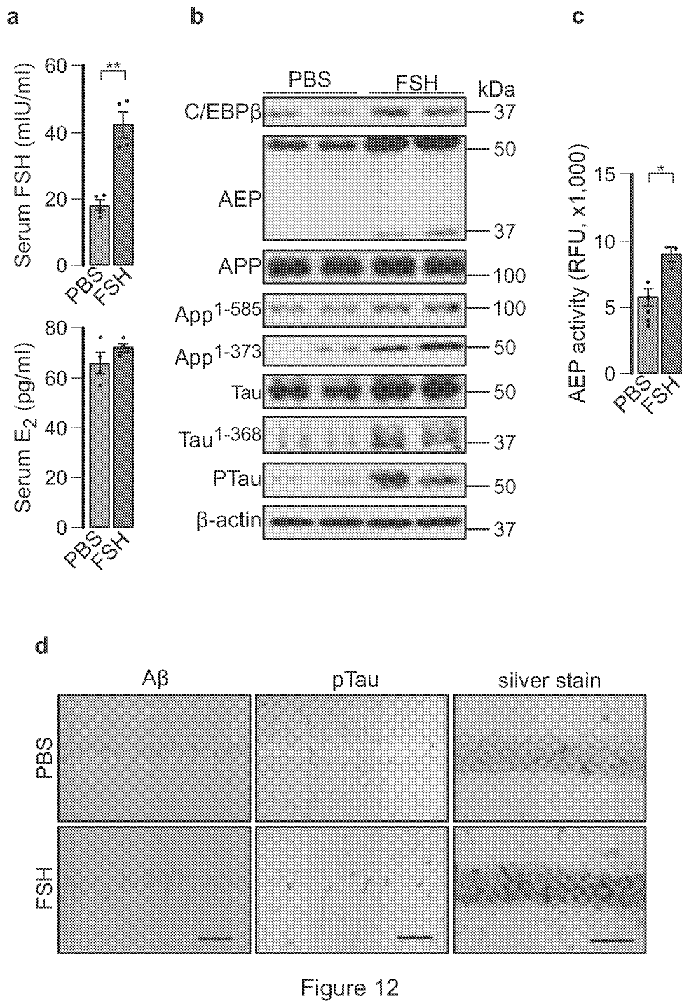

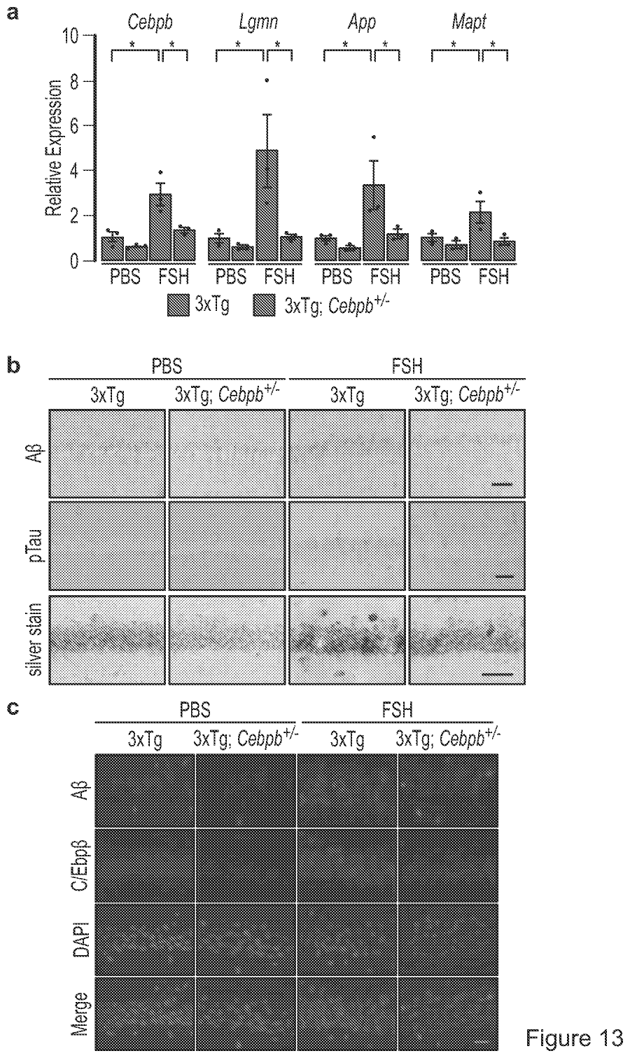

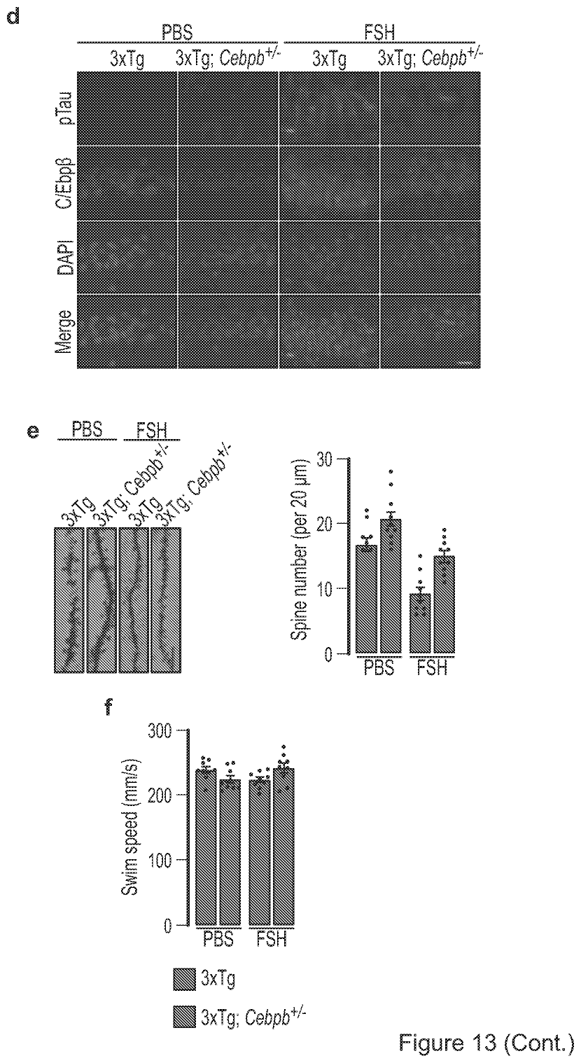

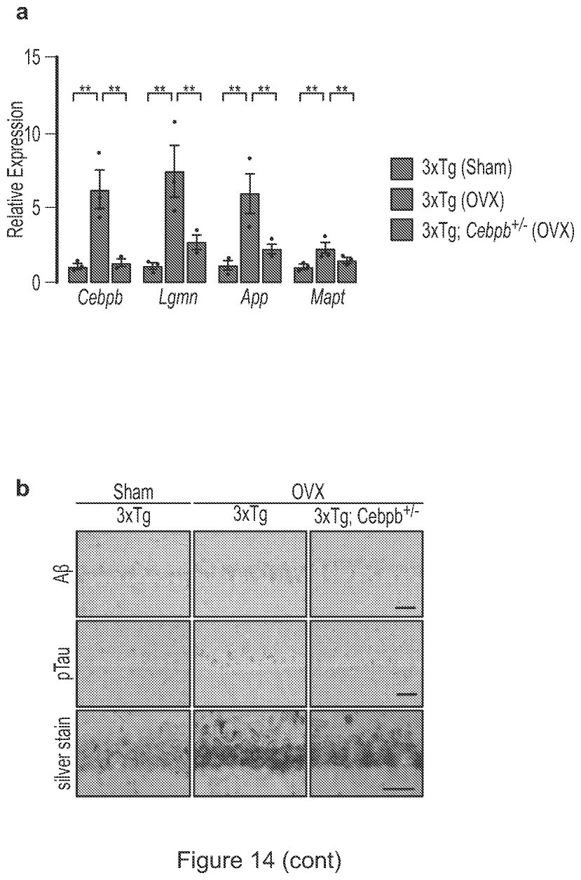

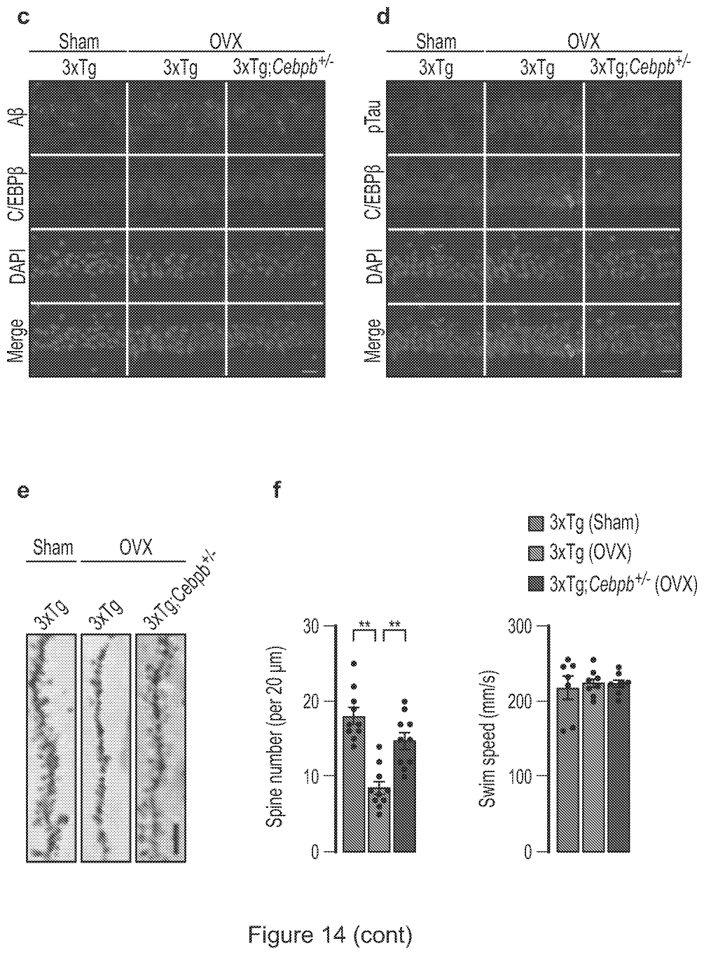

FIG. 6I shows immunofluorescent micrographs showing MAP2, Biotin, DAPI, and merged images of all three markers in brain sections of peripherally injected (i.p.) biotinylated anti-FSHP antibody (FSH Ab) (red) and biotinylated goat IgG. Scale bar, 20 m.

FIG. 6J shows a graph showing Discrimination Index [(Novel Object Head Time—Familiar Object Head Time)/Total Head Time], determined from a Novel Object Recognition test done on APP/PS1 and non-transgenic mice at 9 months of age. IgG (left column) and FSH Ab (right column). Mean±SEM, two-tailed Student's t-test; N=4-10 mice per group).

FIG. 7A shows Western immunoblots showing the effect of activating neuronal FSHRs by FSH (30 ng/mL) in SH-SY5Y cells on levels of total C/EBPβ, phosphorylated C/EBPβ (pC/EBPβ), asparagine endopeptidase (AEP), cleaved amyloid precursor protein (APP) and Tau using antibodies.

FIG. 7B shows relative expression of Cebpb, Lgmn, App and Mapt as measured by quantitative PCR at the indicated time points post treatment in SH-SY5Y cells treated with FSH (30 ng/ml). Mean±SEM; N=3.

FIG. 7C shows a graph depicting AEP enzymatic activity (relative fluorescence units×1000) at the indicated times in the brains of SH-SY5Y cells treated with FSH (30 ng/ml). Mean±SEM, N=4 biological replicates per group. One-way ANOVA; *P<0.05, **P<0.01 compared to zero-hour-FSH group.

FIG. 7D shows relative expression of inflammatory cytokines IL-6, TNF-α and IL-1β as measured by quantitative PCR at the indicated time points post treatment in SH-SY5Y cells with FSH (30 mg/ml). Mean±SEM; N=4; One-way ANOVA; *P<0.05, **P<0.01 compared to zero-hour-FSH group.

FIG. 7E shows Western immunoblots of levels of C/EBPβ, AEP/δ-secretase, APP, cleaved APP¹⁻⁵⁸⁵, total Tau, and cleaved Tau¹⁻³⁶⁸ in response to FSH or PBS following transfection with control (scrambled) or Fshr siRNA.

FIG. 7F shows relative expression of mRNA levels of Fshr, Cebpb, Lgmn, App and Mapt in SH-SY5Y cells incubated with PBS (left column) or FSH (right column) after control or Fshr siRNA transfection. Mean±SEM; three biological replicates; one-way ANOVA; *P<0.05, **P<0.01 compared to PBS+ control siRNA group.

FIG. 7G shows AEP/δ-secretase activity after incubation with PBS (left column) or FSH (30 ng/ml; right column) in control or Fshr siRNA-transfected SH-SY5Y cells. Mean±SEM; three biological replicates; one-way ANOVA; *P<0.05, **P<0.01 compared to PBS+ control siRNA group.

FIG. 7H shows relative percentage levels of inflammatory cytokines, IL-6, TNF-α and IL-1β as measured by ELISAs in SH-SY5Y cells incubated with PBS (left column) or FSH (right column) following control or Fshr siRNA transfection. Mean±SEM; three biological replicates; one-way ANOVA; *P<0.05, **P<0.01 compared to PBS+ control siRNA group.

FIG. 8A shows immunofluorescence micrographs of Aβ, APP, APP^{C586}, AEP, Tau^{N368}, GFP, and merged figures as indicated in rat cortical neuron cultures infected with lentivirus containing shRNA-Cebpb (shCebpb) or adeno-associated virus bearing inactive AEP^{C189S}, and treated with PBS or FSH (30 ng/ml) as indicated. Scale bar, 20 m.

FIG. 8B shows AEP/δ-secretase activity (relative fluorescence units×1000) in rat cortical neuron cultures infected with lentivirus containing shRNA-Cebpb (shCebpb) or adeno-associated virus bearing inactive AEP^{C189S} treated with PBS (left column) or FSH (30 ng/ml; right column) as indicated. Mean±SEM; three biological replicates; one-way ANOVA; *P<0.05, **P<0.01.

FIG. 8C shows Western immunoblots showing the time course of FSH effects on C/EBPβ, phosphorylated C/EBPβ (p C/EBPβ), AEP/δ-secretase, pAEP^{S226}, total AKT, pAKT^{S473}, total ERK1/2, pERK1/2, total SRPK2, pSRPK2^{T492} and pNFκB-p65 (p-p65) expression. Statistics: mean±SEM; three biological replicates; one-way ANOVA; *P<0.05, **P<0.01.

FIG. 9A shows relative expression of Fshr, Cebpb, Lgmn, App and Mapt as measured by quantitative PCR. Sh-Control (left column per group) and sh-Fshr (right column per group) N=3 biological replicates, two-tailed Student's t-test, *P<0.05, **P<0.01.

FIG. 9B shows immunohistochemistry of Aβ and pTau (AT8) in the hippocampus as well as neurofibrillary tangles (Silver staining) in shFshr-injected OVX mice (scale bar, 50 μm).

FIG. 9C shows the fold change in expression as measured by ELISA of Aβ40 and Aβ42 isoforms in sh-Control (left column per group) and shFshr (right column per group)-injected mice.

FIG. 9D shows Golgi-stained dendritic spines in mice with stereotactic shFshr injections (scale bar, 5 m). sh-Control (left column per group) and shFshr (right column per group). N=3 biological replicates; two-tailed Student's t-test, *P<0.05, **P<0.01.

FIG. 10A shows immunofluorescence micrographs showing Aβ (red), cleaved APP^{C586} (green), DAPI (blue) and merged overlays (Merge) as indicated in sections of the hippocampus of male and female 3xTg mice post treatment with PBS (control) or recombinant FSH injected at 5 IU per mouse daily, i.p. for 3 months. Scale bar, 20 μm.

FIG. 10B shows immunofluorescence micrographs showing pTau (AT8, red) and cleaved Tau¹⁻³⁶⁸, DAPI (blue) and merged overlays (Merge) as indicated in sections of the hippocampus of male and female 3xTg mice post treatment with PBS (control) or recombinant FSH injected at 5 IU per mouse daily, i.p. for 3 months. Scale bar, 20 μm.

FIG. 10C shows immunofluorescence micrographs showing Aβ (red) and Thioflavin-S(green), DAPI (blue) and merged overlays (Merge) as indicated in sections of the hippocampus of male and female 3xTg mice post treatment with PBS (control) or recombinant FSH injected at 5 IU per mouse daily, i.p. for 3 months. Scale bar, 20 μm.

FIG. 10D shows immunofluorescence micrographs showing NeuN (red) and TUNEL (green), DAPI (blue) and merged overlays (Merge) as indicated in sections of the hippocampus of male and female 3xTg mice post treatment with PBS (control) or recombinant FSH injected at 5 IU per mouse daily, i.p. for 3 months. Scale bar, 20 μm.

FIG. 10E shows immunofluorescence micrographs showing NeuN (red) and TUNEL (green), DAPI (blue) and merged overlays (Merge) as indicated in sections of the cortex of male and female 3xTg mice post treatment with PBS (control) or recombinant FSH injected at 5 IU per mouse daily, i.p. for 3 months. Scale bar, 20 μm.

FIG. 11A shows immunohistochemistry for Aβ or pTau (AT8) in 3xTg male (M) or female (F) mouse hippocampus post-FSH or PBS injection (scale bar, 50 μm).

FIG. 11B shows silver staining of the prefrontal cortex, and hippocampus CA1 and dorsal ganglion (DG) regions showing amyloid deposits in FSH-injected or PBS-injected male (M) or female (F) 3xTg mice (scale bar, 50 μm).

FIG. 11C shows relative expression of brain mRNA levels of Cebpb, Lgmn, App and Mapt as measured by quantitative PCR in male (M) or female (F) 3xTg mice treated with PBS (left column per group) or FSH (right column per group). N=3; two-tailed Student's t-test, *P<0.05, **P<0.01.

FIG. 11D shows Golgi staining of brain sections from the CA1 region of the hippocampus and the corresponding spine numbers in PBS-(left column per group) or FSH-(right column per group) injected male (M) or female (F) 3xTg mice. Scale bar, 5 μm; N=10 sections.

FIG. 11E shows transmission electron micrographs of hippocampal sections showing synapses (red arrows) post PBS (left column per group) or FSH treatment (right column per group) (scale bar, 1 m). Mean±SEM, N=10 sections; two-tailed Student's t-test, *P<0.05, **P<0.01.

FIG. 12A shows serum levels of FSH (mIU/ml) and 17β-estradiol (E₂; pg/ml) as measured by ELISAs in ovariectomized but estrogen-replete mice treated with PBS (left column) or FSH (right column). 3xTg mice were ovariectomized at 3 months and supplemented with 17β-estradiol using 90-day-release pellets (E₂, 0.36 mg) to render them biochemically eugonadal. The mice were randomly divided to be injected with PBS (OVX+E₂+PBS) or recombinant human FSH (5 IU per mouse daily, i.p.) for 3 months (OVX+E₂+FSH). N=4, two-tailed Student's t-test, *P<0.05, **P<0.01.

FIG. 12B shows western immunoblots of levels of C/EBPβ, AEP/δ-secretase, cleaved APP¹⁻³⁷³ and APP¹⁻⁵⁸⁵, total Tau, cleaved Tau¹⁻³⁶⁸, pTau and β-actin (control) in the brains of ovariectomized but estrogen-replete mice after PBS or FSH injection. kDa=kilodalton.

FIG. 12C shows AEP/δ-secretase enzymatic activity (relative fluorescence units ×1000) in the brains of ovariectomized but estrogen-replete mice after PBS (left column) or FSH (right column) injection. kDa=kilodalton. N=3, two-tailed Student's t-test, *P<0.05, **P<0.01.

FIG. 12D shows expression of Aβ and pTau (AT8) by immunohistochemistry in the hippocampus in ovariectomized but estrogen-replete mice after PBS or FSH injection. Silver staining indicates proteinaceous deposits. Scale bar, 50 μm. Mean±SEM, N=4 (a) and 3 (c), two-tailed Student's t-test, *P<0.05, **P<0.01.

FIG. 13A shows relative expression of Cebpb, Lgmn, App and Mapt mRNA as measured by quantitative PCR following PBS or FSH injection as indicated to 3xTg (left column per group) or 3xTg;Cebpb^{+/-} (right column per group) mice. Mean±SEM, N=3 biological replicates, one-way ANOVA, *P<0.05, **P<0.01.

FIG. 13B shows immunohistochemistry for Aβ and pTau (AT8) and silver staining for amyloid plaques (scale bar, 50 μm) in brain sections from 3xTg or 3xTg;Cebpb^{+/-} mice treated with PBS or FSH.

FIG. 13C shows immunofluorescence staining for Aβ (red), C/EBPβ (green), DAPI (blue) and a merged overlay (Merge) in brain sections from 3xTg or 3xTg;Cebpb^{+/-} mice treated with PBS or FSH. Scale bar, 20 μm.

FIG. 13D shows immunofluorescence staining for pTau (red) and C/EBPβ (green), DAPI (blue) and a merged overlay (Merge) in brain sections from 3xTg or 3xTg;Cebpb^{+/-} mice treated with PBS or FSH. Scale bar, 20 μm.

FIG. 13E shows Golgi staining in the hippocampus in female 3xTg or 3xTg;Cebpb^{+/-} mice, and the graph depicts corresponding dendritic spine numbers post PBS or FSH as indicated for 3xTg (left column per group) or 3xTg;Cebpb^{+/-} (right column per group) mice. Scale bar, 5 m. Mean±SEM, N=10 sections, one-way ANOVA, **P<0.01.

FIG. 13F shows swim speed (mm/s) in a Morris water maze for 3xTg or 3xTg;Cebpb^{+/-} mice treated with PBS (left column) or FSH (right column). N=9 mice/group, one-way ANOVA, *P<0.05, **P<0.01.

FIG. 14A shows relative expression of Cebpb, Lgmn, App and Mapt mRNA as measured by quantitative PCR expression in 3xTg/sham-operated mice (left column), 3xTg/ovariectomized mice (middle column), or 3xTg;Cebpb^{+/-} ovariectomized mice (right column). Mean±SEM, N=3 biological replicates, one-way ANOVA, *P<0.05, **P<0.01.

FIG. 14B shows immunohistochemistry for Aβ and pTau (AT8) and silver staining for amyloid plaques in brain sections of sham or ovariectomized 3xTg mice, or ovariectomized 3xTg;Cebpb^{+/-} mice. Scale bar, 50 μm.

FIG. 14C shows immunofluorescence staining for Aβ (red), C/EBPβ (green) DAPI (Blue) and merged overlays (Merge) in brain sections of sham or ovariectomized 3xTg mice, or ovariectomized 3xTg;Cebpb^{+/-} mice. Scale bar, 20 μm.

FIG. 14D shows immunofluorescence staining for pTau (AT8; red), C/EBPβ (green) DAPI (Blue) and merged overlays (Merge) in brain sections of sham or ovariectomized 3xTg mice, or ovariectomized 3xTg;Cebpb^{+/-} mice. Scale bar, 20 μm.

FIG. 14E shows Golgi staining in the hippocampus in female 3xTg or 3xTg;Cebpb^{+/-} mice and a corresponding graph with dendritic spine numbers (per 20 μm) in sham (left column) or ovariectomized (middle column) 3xTg mice, or ovariectomized 3xTg;Cebpb^{+/-} mice (right column). Scale bar, 5 μm. Mean±SEM, N=10 sections, one-way ANOVA, **P<0.01.

FIG. 14F shows swim speed (mm/s) in a Morris water maze for sham (left column) or ovariectomized (middle column) 3xTg mice, or ovariectomized 3xTg;Cebpb^{+/-} mice (right column). N=7-8 mice/group, one-way ANOVA, *P<0.05, **P<0.01.

Like reference symbols in the various drawings indicate like elements.

### DETAILED DESCRIPTION

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present application will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N Y (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N Y (1998); Coligan et al., Short Protocols in Protein Science, John Wiley & Sons, N Y (2003); Short Protocols in Molecular Biology (Wiley and Sons, 1999).

The nomenclatures used in connection with, and the laboratory procedures and techniques of biochemistry, immunology, microbiology, molecular biology, and virology described herein are those well-known and commonly used in the art.

Throughout this specification and embodiments, the word “comprise,” or variations such as “comprises or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a,” “an,” and “the” are used herein to refer to one or to more than one (i.e., to at least one) of the grammatical object of the article. By way of example, an element means one element or more than one element. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of ±10% within the range of the significant digit.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the Markush group or other grouping of alternatives.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference in their entireties.

The term “about” refers to a range of values that would not be considered by a person of ordinary skill in the art as substantially different from the baseline values. For example, the term “about” may refer to a value that is within 20%, 15%, 10%, 9%, 8%, 7%, 6%, 5%, 4%, 3%, 2%, 1%, 0.5%, 0.1%, 0.05%, or 0.01% of the stated value, as well as values intervening such stated values, for which context will define.

Exemplary methods and materials are described herein, although methods and materials similar or equivalent to those described herein can also be used in the practice or testing of the various aspects and embodiments. The materials, methods, and examples are illustrative only and not intended to be limiting.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

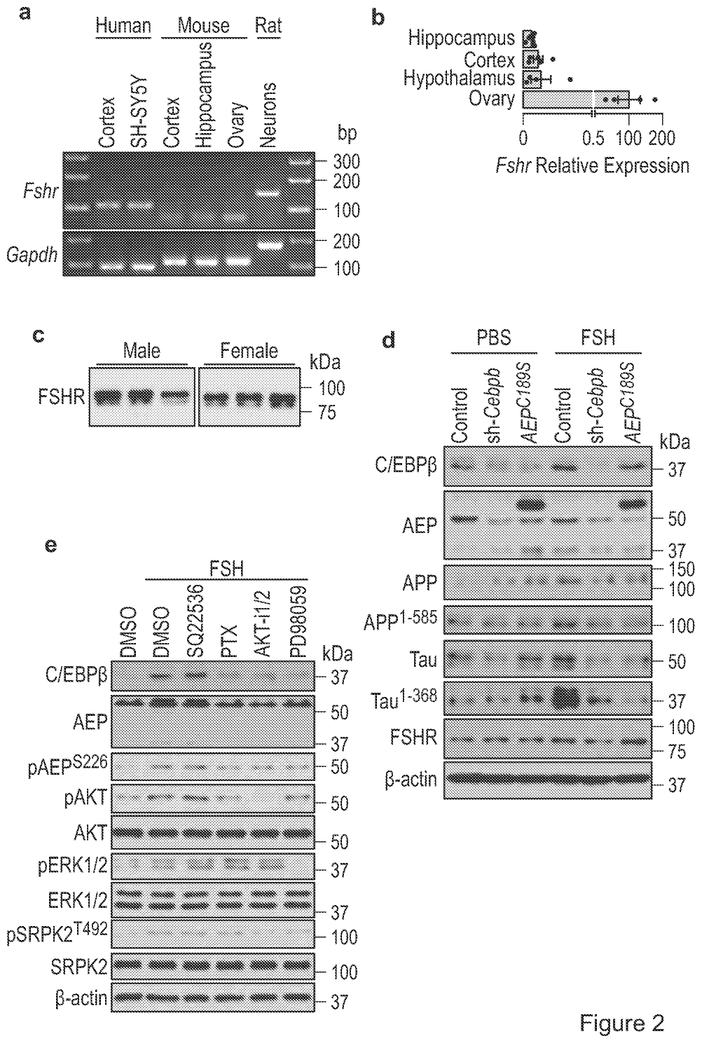

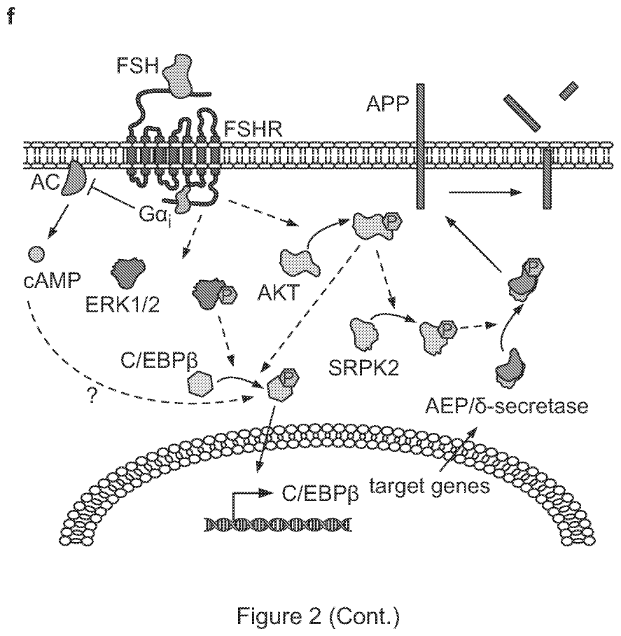

As used herein, the term “neurodegenerative diseases” or “neurodegenerative disorders” refers to a diseases characterized by the deposition of insoluble protein inside and outside brain cells and/or cells of the neuromuscular system. In some embodiments, the disorder is Alzheimer's Disease. Other diseases include but are not limited to progressive supranuclear palsy, frontotemporal lobar degeneration (Pick's disease), corticobasal degeneration and post-encephalitic parkinsonism, frontotemporal dementia with parkinsonism-17 (FTDP-17), argyrophilic grain dementia, British type amyloid angiopathy, cerebral amyloid angiopathy, corticobasal degeneration, CreutzfeldtJakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-StrausslerScheinker disease, Hallervorden-Spatz disease, inclusion body myositis, multiple system atrophy, myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, progressive supranuclear palsy, subacute sclerosing panencephalitis, tangle only dementia, multi-infarct dementia and ischemic stroke; see for a review, e.g., Lee et al., Annu. Rev. Neurosci. 24 (2001), 1121-1159 in which Table 1 catalogs the unique members of tauopathies or Sergeant et al., Bioch. Biophy. Acta 1739 (2005), 179-97, with a list in FIG. 2 therein). AD, the most common cause of dementia, is diagnosed by extracellular plaques containing β-amyloid (Aβ) peptides and intracellular neurofibrillary tangles (NFTs) from hyperphosphorylated, insoluble and filamentous tau proteins in neuropathology (Gao Y. L. et al., Ann. Transl. Med. 2018 May; 6(10): 175). In some embodiments, the disease is an amyloidosis-associated condition or Lewy Body Dementia. In other embodiments, the disease is Parkinson's Disease or Huntingtin disease (HD), which share as a hallmark, protein aggregates with fibrillary amyloid-like structures in the brain. These amyloid fibrils are composed of aggregation-prone proteins, such as mutant huntingtin (HTT) in Huntington disease, and α-synuclein in Parkinson disease (Stroo E et al., Front. Neurosci., 14 Feb. 2017, 11:64).

As used herein, the term “antibody” (Ab) is used in the broadest sense and specifically may include any immunoglobulin, whether natural or partly or wholly synthetically produced, including but not limited to monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2a, IgG2b, IgG3, IgG4, IgM, IgA1, IgA2, IgD, and IgE) and biologically relevant fragment or specific binding member thereof, including but not limited to Fab, F(ab′)₂, scFv (single chain or related entity) and (scFv)₂.

As used herein, the term “antibody fragments” may include those antibody fragments obtained using techniques readily known and available to those of ordinary skill in the art, as reviewed herein. Therefore, in addition to the definition for “antibody” presented supra, the term “antibody” may further encompass any polypeptide or protein comprising a portion of an intact antibody, such as the antigen binding or variable region of the intact antibody. These can be derived from natural sources, or they may be partly or wholly synthetically produced. Examples of antibody fragments include, but are not limited to, Fab, Fab′, F(ab′)2, and Fv fragments; diabodies, and linear antibodies.

The terms “specific binding,” “selective binding,” “selectively binds,” and “specifically binds,” may refer to antibody binding to an epitope on a predetermined antigen but not to other antigens. Typically, the antibody binds with an equilibrium dissociation constant (K_{D}) of approximately less than 106 M, such as approximately less than 10⁻⁷ M, 10⁻⁸ M, 10⁻⁹ M or 10⁻¹⁰ M or even lower when determined by, e.g., equilibrium dialysis or surface plasmon resonance (SPR) technology in a BIACORE® 2000 surface plasmon resonance instrument using the predetermined antigen, e.g., an epitope on FSH, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

As used herein, the term “epitope” may refer to the region of an antigen to which an antibody or T cell binds, e.g. a region within the beta (β) subunit of FSH, including but not limited to an epitope within SEQ ID NO: 1 and/or SEQ ID NO: 2. An “antigen” refers to a substance that elicits an immunological reaction or binds to the products of that reaction.

As used herein, the terms “follicle stimulating hormone” and/or “FSH” may refer to a gonadotropin, a type of glycoprotein polypeptide hormone. FSH is synthesized and secreted by the gonadotropic cells of the anterior pituitary gland and is implicated in regulating the development, growth, maturation, and reproductive processes of the body. FSH is a 35.5 kDa glycoprotein heterodimer, having two polypeptide units, an alpha (α) and beta (β) subunit. FSH is similar in structure to luteinizing hormone (LH), thyroid-stimulating hormone (TSH), and human chorionic gonadotropin (hCG), sharing an identical alpha (α) subunit, but having variations in the beta (β) subunit. This makes the beta (β) subunit an attractive therapeutic target for FSH inhibitors as the inhibitors targeting the beta (β) subunit, e.g. one or more epitopes located within the beta (β) subunit, can be specific to inhibiting FSH. An exemplary gene encoding the beta (β) subunit of human FSH may be accessed at, e.g., Accession No. NM_000510. An exemplary gene encoding the beta (β) subunit of murine FSH may be accessed at, e.g., NM_008045. One of ordinary skill in the art will be able to reach predicted amino acid sequences from the provided nucleotide sequences.

As used herein, the term “FSH inactivating agent” is defined as an agent that reduces the bioactivity or bioavailability of FSH. The FSH inactivating agent may achieve the reduction by binding directly or indirectly to FSH, such as in the case of anti-FSH antibodies or similar peptides or molecules, or the agent may prevent release of FSH from the anterior pituitary gland. In some embodiments, the FSH inactivating agent is a small molecule or a biologic (e.g., a soluble FSH receptor that can bind FSH and block its action). In the case of siRNAs, FSH inactivating agents may prevent translation of mRNA encoding FSH.

As used herein, the term “carriers” may include pharmaceutically acceptable carriers, excipients, or stabilizers that are nontoxic to the cell or mammal being exposed thereto at the dosages and concentrations employed. Often the pharmaceutically acceptable carrier is an aqueous pH-buffered solution. Examples of physiologically acceptable carriers include, but not limited to, buffers such as phosphate, citrate, and other organic acids; antioxidants including, but not limited to, ascorbic acid; low molecular weight (less than about 10 residues) polypeptide; proteins, such as, but not limited to, serum albumin, gelatin, or immunoglobulins; hydrophilic polymers such as, but not limited to, polyvinylpyrrolidone; amino acids such as, but not limited to, glycine, glutamine, asparagine, arginine or lysine; monosaccharides, disaccharides, and other carbohydrates including, but not limited to, glucose, mannose, or dextrins; chelating agents such as, but not limited to, EDTA; sugar alcohols such as, but not limited to, mannitol or sorbitol; salt-forming counterions such as, but not limited to, sodium; and/or nonionic surfactants such as, but not limited to, TWEEN; polyethylene glycol (PEG), and Pluronics. Any combination of such components, including probable inclusion of a bacteriostat, may be useful to fill the formulations of the present disclosure.

As used herein, the term “treating” or “treatment” of a disease may refer to executing a protocol, which may include administering one or more drugs to a patient (human or otherwise), in an effort to alleviate signs or symptoms of the disease. Thus, in the case of treating neurodegenerative diseases (such as Alzheimer's disease), “treating” or “treatment” refers to taking steps to obtain beneficial or desired results, including clinical results, preventing the onset of the disease, reducing cognitive or functional decline in the disease, or enhancing cognitive function. Beneficial or desired clinical results include, but are not limited to, improving cognitive function, delaying or slowing the progression of cognitive impairment, reducing the rate of decline of cognitive function, preventing or slowing the progression of the disease or disorder, or alleviation, amelioration, or slowing the progression, of one or more symptoms associated with the disease. “Treating the disease also includes altering the levels of one of more pathogenic molecules in the disease, including but not limited to, reducing Aβ accumulation; reducing amyloid plaques; and reducing Tau accumulation in the brain.

The term “homology” as used herein may refer to the existence of shared structure between two compositions. The term “homology” in the context of proteins may refer to the amount (e.g. expressed in a percentage) of overlap between two or more amino acid and/or peptide sequences. In the context of nucleic acids, the term may refer to the amount (e.g. expressed in a percentage) of overlap between two or more nucleic acid sequences. As used herein, the percent (%) homology between two sequences is equivalent to the percent identity between the two sequences. The percent identity between the two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm. Such homology is well-represented in the art via local alignment tools and/or algorithms, and may include pairwise alignment, multiple sequence alignment methods, structural alignment methods, and/or phylogenetic analysis methods. Where sequences differ in conservative substitutions, the percent sequence identity may be, but not necessarily is, adjusted upwards to correct for the conservative nature of the substitution. Means for making this adjustment are well known to those of skill in the art. Typically, but not necessarily, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1.

The terms “co-administration,” “co-administered,” and “in combination with” as used herein may refer to the administration of at least two agents or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

The terms “conservative sequence modifications” or “conservative substitutions” as used herein may refer to amino acid modifications to a target epitope or antibodies and antigen-binding portions thereof of the disclosure that does not significantly affect or alter the binding characteristics of the anti-FSH antibodies to the epitope(s), including but not limited to SEQ ID NO: 1 and SEQ ID NO: 2. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a target epitope that the anti-FSH antibodies of the disclosure specifically bind to, e.g. epitopes on the beta (β) subunit of FSH for some anti-FSH antibodies, can be replaced with other amino acid residues from the same side chain family and the antibodies of the present disclosure can be tested against the target epitope can be tested, for example using functional assays described herein or otherwise known in the art.

As used herein, the term “patient” may refer to a biological system to which a treatment can be administered. A biological system can include, for example, an individual cell, a set of cells (e.g., a cell culture), an organ, a tissue, or a multi-cellular organism. A “patient” can refer to a human patient or a non-human patient. In some embodiments, the term “patient” means a female patient. In some embodiments, the term “patient” means a male patient. In some embodiments, the term “patient” means a menopausal female patient. In some embodiments, the term “patient” means a premenopausal female patient. In some embodiments, the term “patient” means a perimenopausal female patient.

As used herein, the terms “purified” or “isolated” antibody, peptide, polypeptide, or protein may refer to a peptide, polypeptide, or protein, which has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein (e.g., anti-FSH antibodies) described in the disclosure can be produced by recombinant DNA techniques.

### FSH/FSHR Inhibiting Agents

The present disclosure provides for agents that are capable of binding to and/or inactivating or inhibiting follicle stimulating hormone (FSH) and/or FSH Receptor (FSHR). In some embodiments, the present disclosure provides for a method of treating neurodegenerative disorders in a subject in need thereof comprising administering a therapeutically effective amount of a FSH inhibiting agent. In some embodiments, the neurodegenerative disease (e.g., Alzheimer's Disease) is treated by (a) reducing AR accumulation; (b) reducing amyloid plaques; (c) reducing Tau accumulation in the brain; and/or (d) enhancing cognitive function.

One exemplary type of inhibiting agent is anti-FSH polyclonal antibodies. However, the disclosure is explicitly not limited as such. FSH inhibiting agents may include small molecules, proteins, peptides, nucleic acids, and the like. Nucleic acids encoding FSH are known. Thus, antisense molecules directed to FSH as well as FSH Receptor (FSHR) find use in the disclosure. In addition, Arey et al. have described a novel synthetic molecule capable of inhibiting the action of FSH. This compound (7-[4-[Bis-(2-carbamoyl-ethyl)-amino]-6-chloro-(1,3,5)-triazin-2-ylamino)-4-hydroxy-3-(4-methoxy-phenylazo)-naphthalene]-2-sulfonic acid, sodium salt) is a selective, noncompetitive inhibitor of the FSH receptor (FSHR) and is described in more detail (Arey et al, Endocrinology, 2002 October; 143(10):3822-9). In addition, U.S. Pat. No. 6,426,357 describes a class of small molecule thiazolidinone FSH receptor antagonists. In addition, such agents may be delivered by a fusion construct to a bisphosphonate or like compound to target it to bone or to TAT, a short peptide for intracellular delivery (Becker-Hapak et al., Methods 2001 July; 24(3):247-56; Vocero-Akbani, A. et al., Methods Enzymol. 2001; 332:36-49). In addition U.S. Pat. No. 6,583,179 describes a series of novel substituted aminoalkylamide derivatives that are antagonists of FSH.

In exemplary embodiments, the present disclosure provides for anti-FSH antibodies and antigen-binding fragments thereof. An anti-FSH antibody may take one of numerous forms in the art, as disclosed herein. Antibodies are in part defined by the antigens to which they bind, thus, an “anti-FSH antibody” is any such antibody which specifically binds at least one epitope found on FSH. In some embodiments, the epitope is located in the beta (β) subunit of FSH. In some embodiments, the epitope is located within LVYKDPARPKIQK (SEQ ID NO: 1). In some embodiments, the epitope is located within LVYKDPARPNTQK (SEQ ID NO: 2). It is understood in the art that an antibody is a glycoprotein comprising at least two heavy (H) chains and two light (L) chains inter-connected by disulfide bonds, or an antigen binding portion thereof. A heavy chain comprises a heavy chain variable region (V_{H}) and a heavy chain constant region (CH1, CH2 and CH3). A light chain comprises a light chain variable region (V_{L}) and a light chain constant region (C_{L}). The variable regions of both the heavy and light chains comprise framework regions (FWR) and complementarity determining regions (CDR). The four FWR regions are relatively conserved while CDR regions (CDR1, CDR2 and CDR3) represent hypervariable regions and are arranged from NH₂ terminus to the COOH terminus as follows: FWR1, CDR1, FWR2, CDR2, FWR3, CDR3, FWR4. The variable regions of the heavy and light chains contain a binding domain that interacts with an antigen while, depending of the isotype, the constant region(s) may mediate the binding of the immunoglobulin to host tissues or factors. It is known in the art that it is possible to manipulate monoclonal and other antibodies and use techniques of recombinant DNA technology to produce other antibodies or chimeric molecules that retain the specificity of the original antibody. Such techniques may evolve introducing DNA encoding the immunoglobulin variable region, or CDRs, of an antibody to the constant regions, or constant regions plus framework regions, of a different immunoglobulin.

The antibodies of the disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent includes the FSH polypeptide. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). The immunization protocol may be selected by one skilled in the art without undue experimentation.

### Pharmaceutical Compositions

The compositions of the present disclosure, either alone or in combination, may be used in vitro, ex vivo, and in vivo depending on the particular application. In accordance, the present disclosure provides for administering a pharmaceutical composition comprising a pharmaceutically acceptable carrier and a pharmacologically effective amount of one or more of the subject peptides, or suitable salts thereof. The pharmaceutical composition may be formulated as powders, granules, solutions, suspensions, aerosols, solids, pills, tablets, capsules, gels, topical crémes, suppositories, transdermal patches, etc.

Pharmaceutically acceptable salts are intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts may include organic acids, such as acetic acid, propionic acid, pyruvic acid, maleic acid, succinic acid, tartaric acid, citric acid, benzoic acid, cinnamic acid, salicylic acid, etc.

The pharmaceutically acceptable composition may be in liquid form or solid form. A solid formulation is generally, but not necessarily, lyophilized and brought into solution prior to administration for either single or multiple dosing. The formulations should not be exposed to extreme temperature or pH so as to avoid thermal denaturation. Thus, it may be important to formulate a composition of the present disclosure within a biologically relevant pH range. A solution buffered to maintain a proper pH range during storage is often necessary, especially for liquid formulations stored for longer periods of time between formulation and administration. Typically, both liquid and solid formulations require storage at lower temperatures (usually, but not necessarily, between 2-8° C.) in order to retain stability for longer periods. Formulated compositions, especially liquid formulations, may contain a bacteriostat to prevent or minimize proteolysis during storage, including but not limited to effective concentrations (usually <1% w/v) of benzyl alcohol, phenol, m-cresol, chlorobutanol, methylparaben, and/or propylparaben. A bacteriostat may be contraindicated for some patients. Therefore, a lyophilized formulation may be reconstituted in a solution either containing or not containing such a component.

Additional components may be added to either a buffered liquid or solid formulation, including but not limited to sugars as a cryoprotectant (including but not necessarily limited to polyhydroxy hydrocarbons such as sorbitol, mannitol, glycerol and dulcitol and/or disaccharides such as sucrose, lactose, maltose or trehalose) and, in some instances, a relevant salt (including but not limited to NaCl, KCl or LiCl). Such formulations, especially liquid formulations slated for long term storage, will rely on a useful range of total osmolarity to both promote long term stability at temperature of, e.g. 2-8° C., or higher, while also making the formulation useful for parenteral injection. For example, but not necessarily, an effective range of total osmolarity (the total number of molecules in solution) may be from about 200 mOs/L to about 800 mOs/L. It will be apparent that the amount of a cyroprotectant, such as sucrose or sorbitol, may depend upon the amount of salt in the formulation in order for the total osmolarity of the solution to remain within an appropriate range. Therefore a salt-free formulation may, but not necessarily, contain from about 5% to about 25% sucrose.

Alternatively, a salt free sorbitol-based formulation may, but not necessarily, contain sorbitol within a range from about 3% to about 12%. Salt-free formulations may warrant increased ranges of the respective cryoprotectant in order to maintain effective osmolarity levels. These formulation may also contain a divalent cation (including but not necessarily limited to MgCl₂, CaCl₂ and MnCl₂); and a non-32 ionic surfactant (including but not necessarily limited to Polysorbate-80 (Tween 80®), Polysorbate-60 (Tween 60®), Polysorbate-40 (Tween 40®) and Polysorbate-20 (Tween 20®), polyoxyethylene alkyl ethers, including but not limited to Brij 58®, Brij 35®, as well as others such as Triton X-100®, Triton X 114®, NP40®, Span 85 and the Pluronic series of non-ionic surfactants (e.g., Pluronic 121)). Any combination of such components, including probable inclusion of a bacteriostat, may be useful to fill the antibody-containing formulations of the present disclosure. The compositions of the present disclosure may also be a “chemical derivative”, which describes compositions that contain additional chemical moieties that are not normally a part of the original compound (e.g., pegylation). Such moieties may improve the solubility, half-life, absorption, etc. of the base FSH inactivating agents. Alternatively the moieties may attenuate undesirable side effects of the base molecule or decrease the toxicity of the base FSH inactivating agents.

Suitable formulations may be found in, among others, Remington's Pharmaceutical Sciences, 17^{th} edition, Mack Publishing Co., Philadelphia, Pa., 1985 and Handbook of Pharmaceutical Excipients, 3rd Ed, Kibbe, A. H. ed., Washington D.C., American Pharmaceutical Association, 2000; hereby incorporated by reference in their entirety. The pharmaceutical compositions described herein can be made in a manner well known to those skilled in the art (e.g., by means conventional in the art, including mixing, dissolving, granulating, levigating, emulsifying, encapsulating, entrapping or lyophilizing processes).

Additionally, the FSH inactivating agents may also be introduced or encapsulated into the lumen of liposomes for delivery and for extending life time of the peptide formulations ex vivo or in vivo. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see for example, U.S. Pat. No. 5,833,948).

Cationic lipids are also suitable for forming liposomes. Generally, the cationic lipids have a net positive charge and have a lipophilic portion, such as a sterol or an acyl or diacyl side chain. Preferably, the head group is positively charged. Typical cationic lipids include 1,2-dioleyloxy-3-(trimethylamino)propane; N-[1-(2,3,-ditetradecycloxy)propyl]-N,N-dimethyl-N-N-hydroxyethylammonium bromide; N-[1-(2,3-dioleyloxy)propyl]-N,N-dimethyl-N-hydroxy ethylammonium bromide; N-[1-(2,3-dioleyloxy)propyl]-N,N,N-trimethylammonium chloride; 3-[N-(N′,N′-dimethylaminoethane)carbamoyl]cholesterol; and dimethyldioctadecylammonium. Of particular interest are fusogenic liposomes, which are characterized by their ability to fuse with a cell membrane upon appropriate change in physiological condition or by presence of fusogenic component, particularly a fusogenic peptide or protein. In one aspect, the fusogenic liposomes are pH and temperature sensitive in that fusion with a cell membrane is affected by change in temperature and/or pH (see for example, U.S. Pat. Nos. 4,789,633 and 4,873,089). Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following endocytosis of liposomes (see Mizoue, T. Int. J. Pharm. 237: 129-137 (2002)).

Another form of fusogenic liposomes comprises liposomes that contain a fusion enhancing agent. That is, when incorporated into the liposome or attached to the lipids, the agents enhance fusion of the liposome with other cellular membranes, thus resulting in delivery of the liposome contents into the cell. The agents may be fusion enhancing peptides or proteins, including hemagglutinin HA2 of influenza virus (Schoen, P. Gene Ther. 6: 823-832 (1999)); Sendai virus envelope glycoproteins (Mizuguchi, H. Biochem. Biophys. Res. Commun. 218: 402-407 (1996)); vesicular stomatitis virus envelope glycoproteins (VSV-G) glycoprotein (Abe, A. et al. J Virol 72: 6159-63 (1998)); peptide segments or mimics of fusion enhancing proteins; and synthetic fusion enhancing peptides (Kono, K. et al. Biochim. Biophys. Acta. 1164: 81-90 (1993); Pecheur, E. I. Biochemistry 37: 2361-71 (1998); U.S. Pat. No. 6,372,720).

Liposomes also include vesicles derivatized with a hydrophilic polymer, as provided in U.S. Pat. Nos. 5,013,556 and 5,395,619, hereby incorporated by reference, (see also, Kono, K. et al. J. Controlled Release 68: 225-35 (2000); Zalipsky, S. et al. Bioconjug. Chem. 6: 705-708 (1995)) to extend the circulation lifetime in vivo. Hydrophilic polymers for coating or derivation of the liposomes include polyethylene glycol, polyvinylpyrrolidone, polyvinylmethyl ether, polyaspartamide, hydroxymethyl cellulose, hydroxyethyl cellulose, and the like. In addition, as described above, attaching proteins that bind a cell surface protein which is endocytosed, e.g., capsid proteins or fragments thereof tropic for a particular cell types and antibodies for cell surface proteins which undergo internalization, may be used for targeting and/or facilitating uptake of the liposomes to specific cells or tissues.

Liposomes are prepared by ways well known in the art (see for example, Szoka, F. et al. Ann. Rev. Biophys. Bioeng. 9: 467-508 (1980)). One typical method is the lipid film hydration technique in which lipid components are mixed in an organic solvent followed by evaporation of the solvent to generate a lipid film. Hydration of the film in aqueous buffer solution, preferably containing the subject peptide or nucleic acid, results in an emulsion, which is sonicated or extruded to reduce the size and polydispersity. Other methods include reverse-phase evaporation (see Pidgeon, C. et al. Biochemistry 26: 17-29 (1987); Duzgunes, N. et al. Biochem. Biophys. Acta. 732: 289-99 (1983)), freezing and thawing of phospholipid mixtures, and ether infusion.

In another preferred embodiment, the carriers are in the form of microparticles, microcapsules, microspheres and nanoparticles, which may be biodegradable or non-biodegradable (see for example, Microencapsulates: Methods and Industrial Applications, Drugs and Pharmaceutical Sciences, Vol 73, Benita, S. ed, Marcel Dekker Inc., New York, 1996). The substance may be within the core of the particle or attached to the particle's polymer network. Generally, the difference between microparticles (or microcapsules or microspheres) and nanoparticles may be one of size.

A variety of materials are useful for making microparticles. Non-biodegradable microcapsules and microparticles include, but are not limited to, those made of polysulfones, poly(acrylonitrile-co-vinyl chloride), ethylene-vinyl acetate, and hydroxyethylmethacrylate-methyl-methacrylate copolymers. These are useful for implantation purposes where the encapsulated FSH inactivating agent diffuses out from the capsules. In another aspect, the microcapsules and microparticles are based on biodegradable polymers, preferably those that display low toxicity and are well tolerated by the immune system. These include protein based microcapsulates and microparticles made from fibrin, casein, serum albumin, collagen, gelatin, lecithin, chitosan, alginate or poly-amino acids such as poly-lysine. Biodegradable synthetic polymers for encapsulating may comprise polymers such as polylactide (PLA), polyglycolide (PGA), poly(lactide-co-glycolide) (PLGA), poly(caprolactone), polydioxanone trimethylene carbonate, polyhydroxyalkonates (e.g., poly(β-hydroxybutyrate)), poly(β-ethyl glutamate), poly(DTH iminocarbony (bisphenol A iminocarbonate), poly (ortho ester), and polycyanoacrylate. Various methods for making microparticles containing the subject compositions are well known in the art, including solvent removal process (see for example, U.S. Pat. No. 4,389,330); emulsification and evaporation (Maysinger, D. et al. Exp. Neuro. 141: 47-56 (1996); Jeffrey, H. et al. Pharm. Res. 10: 362-68 (1993)), spray drying, and extrusion methods.

Another type of carrier is nanoparticles, which are generally suitable for intravenous administrations. Submicron and nanoparticles are generally made from amphiphilic diblock, triblock, or multiblock copolymers as is known in the art. Polymers useful in forming nanoparticles include, but are not limited to, poly(lactic acid) (PLA; see Zambaux et al., J. Control Release 60: 179-188 (1999)), poly(lactide-co-glycolide), blends of poly(lactide-co-glycolide) and polycarprolactone, diblock polymer poly(1-leucine-block-1-glutamate), diblock and triblock poly(lactic acid) (PLA) and poly(ethylene oxide) (PEO) (see De Jaeghere, F. et al., Pharm. Dev. Technol.; 5: 473-83 (2000)), acrylates, arylamides, polystyrene, and the like. As described for microparticles, nanoparticles may be non-biodegradable or biodegradable. Nanoparticles may be also be made from poly(alkylcyanoacrylate), for example poly(butylcyanoacrylate), in which the FSH inactivating agent is absorbed onto the nanoparticles and coated with surfactants (e.g., polysorbate 80). Methods for making nanoparticles are similar to those for making microparticles and may include, among others, emulsion polymerization in continuous aqueous phase, emulsification-evaporation, solvent displacement, and emulsification-diffusion techniques (see Kreuter, J. “Nano-particle Preparation and Applications, In Microcapsules and nanoparticles in medicine and pharmacy,” (M. Donbrow, ed.), pg. 125-148, CRC Press, Boca Rotan, Fla., 1991).

Hydrogels are also useful in delivering the FSH inactivating agents into a host. Generally, hydrogels are cross-linked, hydrophilic polymer networks permeable to a wide variety of drug compounds, including peptides. Hydrogels have the advantage of selective trigger of polymer swelling, which results in controlled release of the entrapped drug compound. Depending on the composition of the polymer network, swelling and subsequent release may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide), poly(N-isopropylacrylamide); poly(methacrylic acid-g-polyethylene glycol); polyacrylic acid and poly(oxypropylene-co-oxyethylene) glycol; and natural compounds such as chrondroitan sulfate, chitosan, gelatin, or mixtures of synthetic and natural polymers, for example chitosan-poly(ethylene oxide).

The gel polymers may be acrylic acid polymers, preferably carbomers (e.g., carboxypolymethylene), such as Carbopol (e.g. Carbopol 420-430, 475, 488, 493, 910, 934P, 974P, and the like; Brock et al., Pharmacotherapy 14: 430-437 (1994)), which are non-linear polymers of acrylic acid cross linked with polyalkenyl polyether. Other types of carbomers include acrylic acids cross-linked with polyfunctional compounds, such as polyallysucrose. In addition to the advantage of hydrating and swelling to a gel, which entraps the subject compounds and limits their release, carbomer gels are mucoadhesive.

For the purposes of this disclosure, the method of administration is chosen depending on the condition being treated, the form of the subject compositions, and the pharmaceutical composition. Administration of the FSH inactivators can be done in a variety of ways, including, but not limited to, cutaneously, subcutaneously, intravenously, orally, topically, transdermally, intraperitoneally, intramuscularly, nasally, and rectally (e.g., colonic administration). For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by rectal or topical administration to the affected area. Alternatively, oral administration is used in conjunction with intravenous or parenteral injections.

The delivery systems also include sustained release or long-term delivery methods, which are well known to those skilled in the art. By “sustained release” or “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the subject peptide, such as biodegradable polymers described above; pumps, including peristaltic pumps and fluorocarbon propellant pumps; osmotic and mini-osmotic pumps; and the like. Peristaltic pumps deliver a set amount of drug with each activation of the pump, and the reservoir can be refilled, preferably percutaneously through a port. A controller sets the dosage and can also provide a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating a disease condition, especially those manifesting recurring episodes or which are progressive in nature, by delivering the FSH inactivating agents of the disclosure via systemic (e.g., intravenous or subcutaneous) or localized doses (e.g., intracerebroventricular) in a sustained, long term manner.

In one preferred embodiment, the method of administration is by oral delivery, in the form of a powder, tablet, pill, or capsule. Pharmaceutical formulations for oral administration may be made by combining one or more peptide with suitable excipients, such as sugars (e.g., lactose, sucrose, mannitol, or sorbitol), cellulose (e.g., starch, methyl cellulose, hydroxylmethyl cellulose, carboxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phthalate, polyvinyl acetate phthalate, hydroxypropyl methyl cellulose phthalate, and the like. Alternatively, oral formulations of the compositions are in prepared in a suitable diluent. Suitable diluents include various liquid form (e.g., syrups, slurries, suspensions, etc.) in aqueous diluents such as water, saline, phosphate buffered saline, aqueous ethanol, solutions of sugars (e.g. sucrose, mannitol, or sorbitol), glycerol, aqueous suspensions of gelatin, methyl cellulose, hydroxylmethyl cellulose, cyclodextrins, and the like. As used herein, diluent or aqueous solutions also include infant formula. In some embodiments, lipohilic solvents are used, including oils, for instance vegetable oils, peanut oil, sesame oil, olive oil, corn oil, safflower oil, soybean oil, etc.); fatty acid esters, such as oleates, triglycerides, etc.; cholesterol derivatives, including cholesterol oleate, cholesterol linoleate, cholesterol myristilate, etc.; liposomes; and the like.

In one embodiment, administration is done rectally. This may use formulations suitable for topical application in the form of salves, tinctures, cremes, or for application into the lumen of the intestine by use of compositions in the form of suppositories, enemas, foams, etc. Suppositories may contain conventional suppository bases such as cocoa butter, carbowaxes, polyethylene glycols, or glycerides, which are solid or semi-solid at room temperature but liquid at body temperature.

In yet another embodiment, the administration is carried out cutaneously, subcutaneously, intraperitoneally, intramuscularly or intravenously. As discussed above, these are in the form of peptides dissolved or suspended in suitable aqueous medium, as discussed above. Additionally, the pharmaceutical compositions for injection may be prepared in lipophilic solvents, which include, but is not limited to, oils, such as vegetable oils, olive oil, peanut oil, palm oil soybean oil, safflower oil, etc.; synthetic fatty acid esters, such as ethyl oleate or triglycerides; cholesterol derivatives, including cholesterol oleate, cholesterol linoleate, cholesterol myristilate, etc.; or liposomes, as described above. The compositions may be prepared directly in the lipophilic solvent or preferably, as oil/water emulsions, (see for example, Liu, F. et al. Pharm. Res. 12: 1060-1064 (1995); Prankerd, R. J. J. Parent. Sci. Tech. 44: 139-49 (1990); U.S. Pat. No. 5,651,991).

The dose and dosage regimen depends upon a variety of factors readily determined by a physician, such as the nature of the infection, for example, its therapeutic index, the patient, and the patient's history. Generally, a therapeutically effective amount of a composition is administered to a patient. In some embodiments, the amount of composition administered is in the range of about 0.001 mg/kg to about 1000 mg/kg of patient body weight, and any range in between. Depending on the severity of condition, about 0.1 mg/kg to about 50 μmg/kg body weight (for example, about 0.1-15 mg/kg/dose, more usually from about 1-25 mg/kg body weight) of composition is an initial candidate dosage for administration to the patient, whether, for example, by one or more separate administrations, or by continuous infusion. The compositions may be delivered relatively low volume rates, for example but not necessarily from about 0.001 ml/day to 10 ml/day so as to minimize tissue disturbance or trauma near the site where the formulation is released. The formulation may be released at a rate of, depending on the specific biological agent(s), at a low dose, e.g., from about 0.01 μg/hr or 0.1 μg/hr, 0.25 μg/hr, 1 μg/hr, generally up to about 200 μg/hr, or the formulation is delivered at a low volume rate e.g., a volume rate of from about 0.001 ml/day to about 1 ml/day, for example, 0.01 micrograms per day up to about 20 milligrams per day. Dosage depends on a number of factors such as potency, bioavailability, and toxicity of the active ingredient and the requirements of the subject. The progress of this therapy is readily monitored by conventional methods and assays and based on criteria known to the physician or other persons of skill in the art. The above parameters for assessing successful treatment and improvement in the disease are readily measurable by routine procedures familiar to a physician.

The delivery systems also include sustained release or long-term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the subject peptide, such as biodegradable polymers described above; pumps, including peristaltic pumps and fluorocarbon propellant pumps; osmotic and mini-osmotic pumps; and the like. Peristaltic pumps deliver a set amount of drug with each activation of the pump, and the reservoir can be refilled, preferably percutaneously through a port. A controller sets the dosage and can also provide a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating an inflammatory disease condition, especially those manifesting recurring episodes or which are progressive in nature, by delivering the FSH inactivators of the disclosure via systemic (e.g., intravenous or subcutaneous) or localized doses in a sustained, long term manner.

The present disclosure also encompasses the therapeutic combinations disclosed herein in the form of a kit or packaged formulation. A kit or packaged formulation as used herein includes one or more dosages of a subject peptide, and salts thereof, in a container holding the dosages together with instructions for simultaneous or sequential administration to a patient. For example, the package may contain the peptides along with a pharmaceutical carrier combined in the form of a powder for mixing in an aqueous solution, which can be ingested by the afflicted subject. The package or kit includes appropriate instructions, which encompasses diagrams, recordings (e.g., audio, video, compact disc), and computer programs providing directions for use of the combination therapy. The foregoing descriptions of specific embodiments of the present disclosure have been presented for purposes of illustration and description. They are not intended to be exhaustive or to limit the disclosure to the precise forms disclosed, and obviously many modifications and variations are possible in light of the above teaching.

Publications disclosed herein are provided solely for their disclosure prior to the filing date of the present disclosure. Nothing herein is to be construed as an admission that the present disclosure is not entitled to antedate such publication. Further, the dates of publication provided may be different from the actual publication dates which may need to be independently confirmed.

Each of the applications and patents cited in this text, as well as each document or reference, patent or non-patent literature, cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself, and, each of these documents or references (“herein-cited references”), as well as each document or reference cited in each of the herein-cited references (including any manufacturer's specifications, instructions, etc.), is hereby expressly incorporated herein by reference.

The following non-limiting examples serve to further illustrate the present disclosure.

### Methods of Treatment

The methods disclosed herein enable the treatment of neurodegenerative disorders (such as Alzheimer's Disease) in subjects with compositions of the disclosure. A composition of the disclosure comprises anti-FSH polyclonal antibodies administered to the human patient. An antibody of the disclosure is a biologic treatment for Alzheimer's disease which specifically binds to one or more epitopes within a β-subunit of FSH comprising SEQ ID NO: 1 or SEQ ID NO: 2 or a peptide sequence consisting essentially of SEQ ID NO: 1 or SEQ ID NO: 2 but having conservative substitutions.

The methods of this disclosure further comprise administration of an FSH inhibiting agent (e.g., an anti-FSH antibody) in combination with an additional therapeutic agent for a neurodegenerative disease (e.g., AD). In some embodiments, an antibody of the disclosure is concurrently administered with one or more agents including but not limited to cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of NGF, CERE-110, beta-blocker, human amyloid vaccine, beta or gamma secretase inhibitor, nicotinic or muscarinic agonist, and a second antibody. The cholinesterase inhibitor includes but is not limited to galantamine, rivastigmine, tacrine, and donepezil. The NMDA receptor antagonist includes but is not limited to ketamine, methadone, memantine, amantadine, and dextromethorphan or a salt thereof. The antipsychotic agent includes but is not limited to aripiprazole, risperidone, olanzapine, quetiapine, or haloperidol. The benzodiazepine includes but is not limited to lorazepam, oxazepam and temazepam. The tricyclic antidepressant includes but is not limited to nortriptyline. The hormone includes but is not limited to estrogen, progesterone and leuprolide. The vitamin includes but is not limited to folate and nicotinamide. The second antibody includes but is not limited to bapineuzumab, solanezumab, gantenerumab, crenezumab, ponezumab, BAN2401, and aducanumab.

Furthermore, any therapy described herein can include one or more agents for treating, one or more side-effects of a therapy comprising the neurodegenerative disease therapy. Combination therapies (e.g., co-administration of an anti-FSH antibody composition and one or more additional neurodegenerative therapies or additional therapeutic agents) can be, e.g., simultaneous or successive. For example, the FSH inhibiting agent (e.g., anti-FSH antibody) of this disclosure and the additional therapeutic agent(s) can be administered at the same time or at different times. In some embodiments, the one or more additional therapeutic agents can be administered first in time and the FSH inhibiting agent can be administered second in time.

### EXAMPLES

The practice of the methods and compositions of the disclosure employs, unless otherwise indicated, conventional techniques of molecular biology (including recombinant techniques), cell culture, microbiology, cell biology, biochemistry, immunology, and neuroscience, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The following materials, reagents, and methods are used for the Examples described herein.

### Transgenic Mice

3xTg mice were obtained from Jackson Laboratory (stock #34830). The mice harbor a human transgene with mutated APP^{K670N/M671L} and MAPT^{P301L}, and a knock-in mutation Psen1^{M146V} and display AD neuropathology and cognitive decline at about 3 to 4 months (Oddo, S. et al. Neuron 39, 409-421, (2003)), which is known to be accelerated upon ovariectomy. Mutant Cebpb mice (Sterneck, E., et al. Genes Dev 11, 2153-2162, (1997)) were maintained at Emory University as heterozygotes on C57BL/6 and 129Sv backgrounds. The two strains were crossed to generate viable F1 hybrid wild type and Cebpb^{+/-} littermates; the latter were then crossed with 3xTg mice to generate compound 3xTg;Cebpb^{+/-} and 3xTg;Cebpb^{+/-} mutants. APP/PS1 mice, which were generated and bred at Mount Sinai, harbor a human transgene comprising APPK^{670N/M671L} and PSEN1^{ΔE9} (Minkeviciene, R. et al. J Neurochem 105, 584-594, (2008)). Unlike 3xTg mice, APP/PS1 mice develop amyloid plaques at 6 months, overt cognitive impairment at a later age of 12 months, and do not display neurofibrillary tangles (Minkeviciene, R. et al. J Neurochem 105, 584-594, (2008); Onos, K. D. et al. PLoS Genet 15, e1008155, (2019); Volianskis, A., et al. Neurobiol Aging 31, 1173-1187, (2010)). These mice, when bred on a pure C57BL/6J background, show evidence of sudden death due to seizure activity (Minkeviciene, R. et al. J Neurosci 29, 3453-3462, (2009)). Animal care and handling were performed according to NIH animal care guidelines at both Emory and Mount Sinai. The protocols were reviewed and approved by the respective Institutional Animal Care and Use Committees.

### Anti-FSH Antibodies

The antibodies used in the experiments set forth in this disclosure were generated against a 13-amino-acid mouse FSHβ sequence, LVYKDPARPNTQK (SEQ ID NO: 2) (Zhu, L. L. et al. Proc Natl Acad Sci USA 109, 14574-14579, (2012)). FSH Ab binds to, and blocks the action of FSHβ on the G protein-coupled FSH receptors (FSHRs) on bone cells and adipocytes, and in doing so, inhibits bone formation, reduces bone resorption, decreases body fat and increases energy expenditure (Zhu, L. L. et al. Proc Natl Acad Sci USA 109, 14574-14579, (2012); Ji, Y et al. Proc Natl Acad Sci USA 115, 2192-2197, (2018); Liu, P. et al. Nature 546, 107-112, (2017); Zhu, L. L. et al. Biochem Biophys Res Commun 422, 54-58, (2012)).

### Stereotactic Injection

AAV2-Fshr virus (iAAV04355302) and control virus (iAAV01502) from Applied Biological Materials Inc. were injected stereotactically into 3-month-old female 3xTg mice under isoflurane anesthesia. The following coordinates were used for bilateral intracerebral injections: −2.1 mm anteroposterior and −1.8 mm mediolateral from the bregma, and −1.5 mm dorsoventral from the dural surface. 2 μl viral suspension containing 2×10⁹ vector genomes/μl was placed into each site at a rate of 0.25 μl/min using 10 μl glass syringes with a fixed needle. The needle remained in place for 5 min and was removed slowly over 2 min. Mice were placed on a heating pad until they turned from a supine to a prone position, indicative of recovery from anesthesia. The mice underwent ovariectomy 7 days after stereotactic injection.

### Antibodies and Reagents

Antibody to C/EBPβ (HT-7) (catalog #: sc-7962) was from Santa Cruz; anti-AEP 6E3 was a gift from Dr. Colin Watts, Professor of Immunobiology, Division of Cell Signaling and Immunology, College of Life Sciences, University of Dundee, Dundee, UK; antibodies to FSHR (catalog #: PA5-50963), Tau^{pSer202-Thr205} (AT8, catalog #: MN1020) were from Thermo Fisher Scientific; antibodies to Legumain (D6S4H) (catalog #: 93627), AKT (catalog #: 4691s), pAKTS473 (rabbit monoclonal, catalog #: 9271s), ERK1/2 (catalog #: 9102s), pERK1/2 (catalog #: 9106s) were purchased from Cell Signaling Technology; antibody to NeuN (catalog #: MAB377), tau 5 (catalog #: MAB361), β-actin (catalog #: A5316) were from Sigma-Aldrich; antibody to Aβ (4G8) (catalog #: 800701) was obtained from Biolegend; and antibody to SRPK2 (catalog #: 611118) were from BD Biosciences. Antibodies to pAEP^{S226}, Tau¹⁻³⁶⁸, APP¹⁻⁵⁸⁵, APP¹⁻³⁷³ and pSRPK2^{T492} were developed in the Ye lab. The anti-FSHβ polyclonal antibody (FSH Ab) was developed and characterized in the Zaidi lab. Fshr expression plasmid (catalog #: 66286) was from Addgene; siRNA-Fshr (catalog #: sc-35415) was from Santa Cruz, and the TUNEL In Situ Cell Death Detection Kit (catalog #: 11684817910) was from Roche. Human Aβ40 (catalog #: KHB3481), Aβ42 (catalog #: KHB3544), and inflammatory cytokine ELISA kits (catalog #: BMS223-4, BMS224-2, KHC0061) were purchased from Invitrogen. The AEP substrate Z-Ala-Ala-Asn-AMC (catalog #: 4033201) was from Bachem, and EZ-Link Sulfo-NHS-LC-Biotinylation Kit was obtained from Thermo Fisher (Catalog #21435). 90-day-release pellets containing 0.36 mg 170-estradiol (catalog #: NE121) were purchased from Innovative Research of America. All chemicals not mentioned above were purchased from Sigma-Aldrich.

### Human Tissue

Post-mortem brain tissue was from the Emory Alzheimer's Disease Research Center. The study was approved by the Biospecimen Committee at Emory University. Informed consent was obtained from all subjects.

### Cells

The human neuroblastoma cell line SH-SY5Y, obtained from ATCC, was cultured in DMEM/F12 with 10% fetal bovine serum (FBS), penicillin (100 units/ml) and streptomycin (100 μg/ml). SH-SY5Y transfection was performed using Lipofectamine 3000 (Invitrogen). Primary culture of rat cortical neurons was described earlier. LV-shCebpb-GFP, LV-GFP and AAV-AEP^{C189S} were packaged by Viral Vector Core (VVC) of Emory University. 2 μl of virus solution was added to 1 ml culture medium and applied to primary neuron cultures. Cells were incubated at 37° C. in a humidified atmosphere of 5% CO₂.

### AEP Activity Assay

Tissue homogenates or cell lysates (10 μg) were incubated in 200 μl assay buffer (20 mM citric acid, 60 mM Na₂HPO₄, 1 mM EDTA, 0.1% CHAPS, and 1 mM DTT, pH 6.0) containing 20 μM δ-secretase substrate, Z-Ala-Ala-Asn-AMC (Bachem). AMC released by substrate cleavage was quantified by measuring at 460 nm in a fluorescence plate reader at 37° C. in kinetic mode.

### Quantitative PCR

mRNA levels were analyzed by quantitative PCR (qPCR). Briefly, RNA was isolated by TRIzol (Life Technologies). Reverse transcription was performed with SuperScript III reverse transcriptase (Life Technologies). Gene-specific primers and probes were designed and bought from Taqman (Life Technologies). All qPCR reactions were performed using the ABI 7500-Fast Real-Time PCR System and the Taqman Universal Master Mix Kit (Life Technologies). The relative quantification of gene expression was calculated using the ΔΔCt method.

### Western Immunoblotting

Cells and brain tissue were washed with ice-cold PBS and lysed in (50 μmM Tris-HCl, pH 7.4, 40 mM NaCl, 1 mM EDTA, 0.5% Triton X-100, 1.5 mM Na₃VO₄, 50 mM NaF, 10 mM sodium pyrophosphate, 10 mM sodium β-glycerophosphate, supplemented with protease inhibitors cocktail) at 4° C. for 0.5 hours, and centrifuged for 25 min at 15,000 rpm. The supernatant was boiled in SDS loading buffer. After SDS-PAGE, the samples were transferred to a nitrocellulose membrane. The membrane was blocked with TBS containing 5% nonfat milk and 0.1% Tween 20 (TBST) at room temperature for 2 hours, followed by incubation with primary antibody at 4° C. overnight, and with the secondary antibody at room temperature for 2 hours. After washing with TBST, the membrane was developed using the enhanced chemiluminescent (ECL) detection system.

### Immunostaining

Free-floating 25-am brain sections were used for immunostaining. For immunohistochemistry, brain sections were treated with 0.3% H₂O₂ for 10 minutes. Sections were washed three times in PBS and blocked in 1% BSA and 0.3% Triton X-100 for 30 min, followed by overnight incubation with anti-AP antibody (1:400), and AT8 (1:200) at 4° C. Signal was developed using Mouse and Rabbit Specific HRP/DAB (ABC) Detection IHC kit (Abcam). For immunofluorescence staining, sections were incubated overnight at 4° C. with primary Aβ (1:400), APPC586 (1:200), AT8 (1:200) or Taul-368 (1:600) antibodies. After washing with Tris-buffered saline, sections were incubated with a mixture of Alexa Fluor 488- and 594-coupled secondary antibodies (Invitrogen) for detection. DAPI (1 μg/mL) (Sigma) was used for staining nuclei. Images were acquired with an Olympus Confocal FV1000 Imaging System.

### Gallyas Silver Staining

Brain sections (25 μm) were incubated in 5% periodic acid for 5 min, washed in water, and then placed in alkaline silver iodide solution (containing 1% silver nitrate) for 1 min. The sections were then washed in 0.5% acetic acid for 10 min, placed in developer solution for 15 min, and washed with 0.5% acetic acid and then with water. The sections were treated with 0.1% gold chloride for 5 min, washed in water, and incubated in 1% sodium thiosulfate (hypo) for 5 min before a final washing.

### Thioflavin-S Staining

Amyloid plaques were stained with Thioflavin-S. Free-floating 25-μm brain sections were incubated in 0.25% potassium permanganate solution for 20 min, rinsed in distilled water, and incubated in bleaching solution containing 2% oxalic acid and 1% potassium metabisulfite for 2 min. After rinsing in distilled water, the sections were transferred to blocking solution containing 1% NaOH and 0.9% H₂O₂ for 20 min. The sections were incubated for 5 sec in 0.25% acetic acid, washed in distilled water, and stained for 5 min with 0.0125% thioflavin-S in 50% ethanol. The sections were washed with 50% ethanol, placed in distilled water, covered with a glass cover using mounting solution, and examined under a fluorescence microscope.

### Golgi Stain

Mouse brains were fixed in 10% formalin for 24 h and then immersed in 3% potassium bichromate for 3 days in the dark. The solution was changed each day and the brains transferred into 2% silver nitrate solution and incubated for 7 days in the dark. The solution was changed each day. Vibratome sections were cut at 30 μm, air dried for 10 min, dehydrated through 95% and 100% ethanol, cleared in xylene, and cover-slipped. Spine numbers were counted as previously.

### Electron Microscopy of Synapse

After deep anesthesia, mice were perfused transcardially with 4% paraformaldehyde in PBS. Hippocampal slices were post-fixed in cold 1% OsO₄ for 1 hour. Samples were prepared and examined using standard procedures. Ultrathin sections (90 nm) were stained with uranyl acetate and lead acetate and viewed at 100 kV in a JEOL 200CX Electron Microscope. Synapses were identified by the presence of synaptic vesicles and postsynaptic densities. Synapse number was quantitated as before.

### Morris Water Maze

Mice were trained in a round, water-filled tub (52 inch diameter) in an environment rich with extra maze cues as described previously. Each subject was given 4 trials/day for 5 consecutive days with a 15-min inter-trial interval. The maximum trial length was 60 sec and, if mice did not reach the platform in the allotted time, they were manually guided to it. Following the 5 day of task acquisition, a probe trial was presented during which time the platform was removed and the percentage of time spent in the quadrant which previously contained the escape platform during task acquisition was measured over 60 sec. All trials were analyzed for latency by means of MazeScan (Clever Sys, Inc.).

### Novel Object Recognition Test

This is a commonly utilized behavioral test, in which a mouse is presented with two identical objects during the first session, and then one of the two objects is replaced by a novel object during a second session (Leger, M. et al. Nat Protoc 8, 2531-2537, (2013)). On day 1, a habituation phase in an empty arena (for 5 minutes), was followed 24 hours later, by the training phase, which allows for a 5-minute exploration in the habituated arena where two identical objects are placed in opposite quadrants. The testing phase followed a gap of 20 minutes, which corresponds to a retention time frame that would normally allow memory deficits to be captured. For testing, one object was replaced with a novel object followed by 5 minutes of exploration. Parameters were collected using an ANY-maze Video Tracking System (Stoelting, UK).

### Electrophysiology

Mice were anaesthetized with isoflurane, decapitated, and their brains dropped in ice-cold artificial cerebrospinal fluid (a-CSF) containing 124 mM NaCl, 3 mM KCl, 1.25 mM NaH₂PO₄, 6.0 mM MgCl₂, 26 mM NaHCO₃, 2.0 mM CaCl₂, and 10 mM glucose. Hippocampi were dissected and cut into 400-mm thick transverse slices with a vibratome. After incubation at 23-24° C. in a-CSF for 60 to 90 min, slices were placed in a recording chamber (RC-22C, Warner Instruments) on the stage of an upright microscope (Olympus CX-31) and perfused at a rate of 3 mL per min with a-CSF (containing 1 mM MgCl₂) at 23 to 24° C. A 0.1 MU tungsten monopolar electrode was used to stimulate the Schaffer collaterals. The field excitatory post-synaptic potentials (fEPSPs) were recorded in CA1 stratum radiatum by a glass microelectrode filled with a-CSF with resistance of 3-4 MU. The stimulation output (Master-8; AMPI, Jerusalem) was controlled by the trigger function of an EPC9 amplifier (HEKA Elektronik, Lambrecht, Germany). fEPSPs were recorded under current-clamp mode. Data were filtered at 3 kHz and digitized at sampling rates of 20 kHz using Pulse software (HEKA Elektronik). Stimulus intensity (0.1 ms duration, 10-30 mA) was set to evoke 40% of the maximum f-EPSP and the test pulse was applied at a rate of 0.033 Hz. LTP of fEPSPs was induced by 3 theta-burst-stimulation (TBS, 4 pulses at 100 Hz, repeated 3 times with a 200-ms interval). The magnitudes of LTP are expressed as the mean percentage of baseline fEPSP initial slope.

### Statistical Analysis

Statistical analyses were performed using either Student's t-test (two-group comparison) or one-way ANOVA followed by Fisher's Least Significant Difference post-hoc test (more than two groups). Differences with P values less than 0.05 were considered significant.

### Example 1: FSH Antibody Attenuates Neuropathology and Cognitive Decline in AD Mice

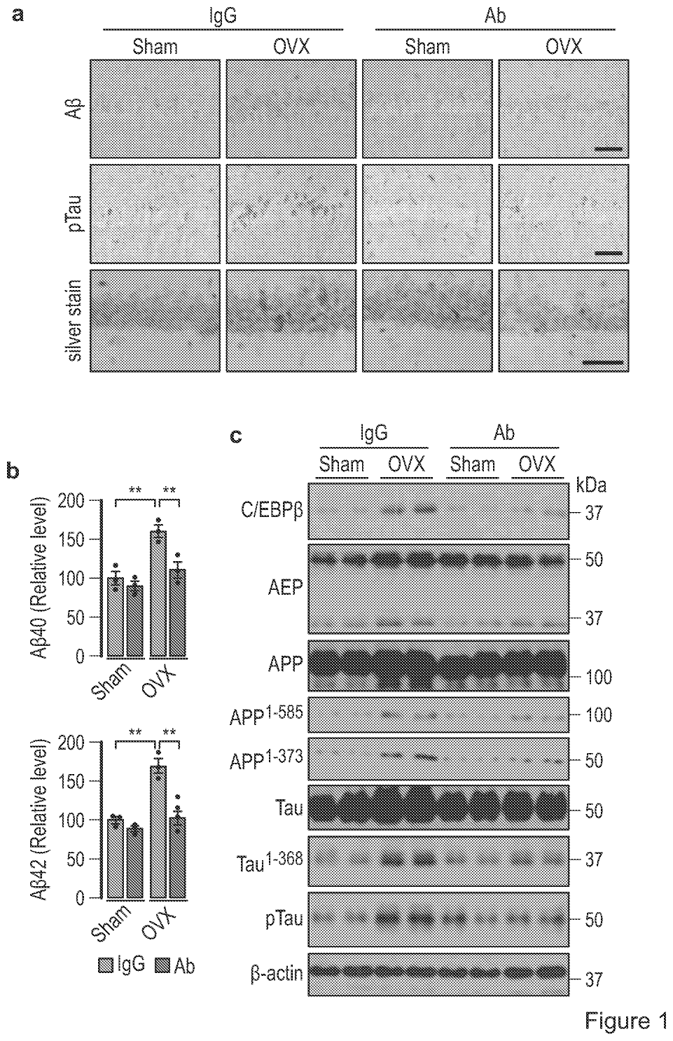

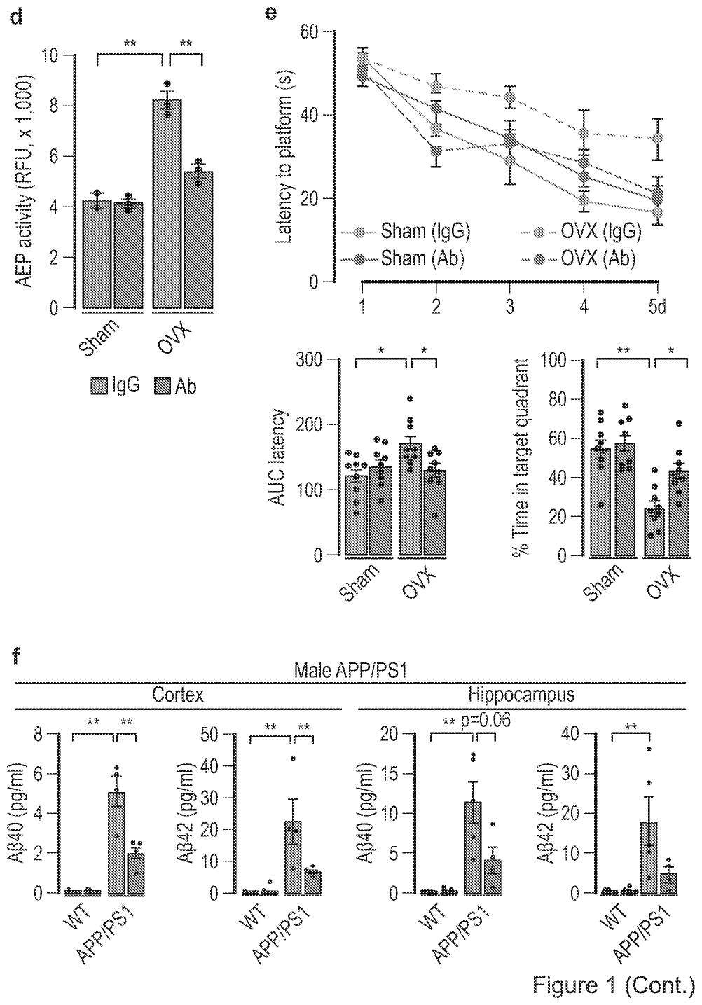

3.5-month-old female 3xTg mice were ovariectomized or sham-operated, 4 days after which the groups were divided to receive FSH Ab or goat IgG (200 μg/mouse, every 2 days, i.p.) for 8 weeks. Ovariectomy expectedly induced uterine atrophy and elevated FSH levels (FIG. 6A). Importantly, however, as noted in previous studies (Zhu, L. L. et al. Proc Natl Acad Sci USA 109, 14574-14579, (2012); Liu, P. et al. Nature 546, 107-112, (2017)), FSH Ab treatment did not alter serum FSH or estrogen levels in sham-operated mice (FIG. 6A). Immunohistochemistry of the hippocampus showed increased Aβ and Tau accumulation post-ovariectomy, which was reduced upon FSH Ab treatment (FIG. 1A and FIG. 11B). The FSH Ab also dramatically reduced the ovariectomy-induced enhancements in hippocampal accumulation of the Aβ40 and Aβ42 isoforms (FIG. 1B).

Ovariectomy strongly induced the expression of the transcription factor C/EBPβ in hippocampal neurons (FIG. 1C, and FIG. 6C). C/EBPβ is known to activate AEP/δ-secretase21 that cleaves amyloid precursor protein (APP) at residues N373 and N585 and Tau at N368, respectively, to promote Aβ and Tau aggregates. Deletion of AEP from 5xFAD or Tau^{P301S} mice is known to ameliorate amyloid plaques and neurofibrillary tangles, respectively, and rescue cognitive deficits (Zhang, Z. et al. Nat Med 20, 1254-1262, (2014); Zhang, Z. et al. Nat Commun 6, 8762, (2015)). Consistent with the increased expression and activation of the C/EBPβ-AEP/δ-secretase pathway (FIG. 1D and FIG. 6C), ovariectomy was found to induce APP and Tau cleavage, and Tau phosphorylation in 3xTg mice (FIG. 1C, and FIG. 6B). Importantly, these effects were reversed with the FSH Ab treatment (FIG. 1C, and FIG. 6B). FSH Ab also reversed the ovariectomy-induced enhancement in neuronal apoptosis and reductions in dendritic spine and synapse numbers (FIGS. 6D-6F).

For functional correlation, behavioral tests were conducted, including Morris Water Maze and Fear Conditioning Tests. Morris Water Maze testing showed that ovariectomy induced a significant learning deficit in 3xTg mice within 2 months, as was evident from increased latency to mount a platform and time spent in the platform quadrant—both phenotypic variables were reversed in FSH Ab-treated ovariectomized mice (FIG. 1E).

There is evidence that post-menopausal LH levels correlate with a higher incidence of AD (Henderson, V. W., et al., Arch Neurol 51, 896-900, (1994); Rocca, W. A. et al. Neurology 69, 1074-1083, (2007)); that LHβ transgenic mice are cognitively impaired (Casadesus, G. et al. Mol Cell Endocrinol 269, 107-111, (2007)); and that hCG induces cognitive deficits in rodents (Berry, A., et al. Horm Behav 54, 143-152, (2008); Barron, A. M., et al. Endocrinology 151, 5380-5388, (2010)). However, the highly FSH-specific Ab used for these experiments does not cross-react with LH (FIG. 6H), suggesting that FSH reductions are largely responsible for the rescue of AD pathology in 3xTg mice post-ovariectomy. That the FSH Ab crossed the blood-brain barrier was further confirmed by injecting both biotinylated FSH Ab and goat IgG, i.p., into wild type mice. Immunofluorescent co-staining with MAP2 and DAPI showed a non-cellular localization of both molecules in whole brain sections, consistent with the extracellular inhibition of FSH action on the hippocampal FSHR (see below) (FIG. 6I).

The data obtained using 3xTg mice was replicated in a different mouse model—the APP/PS1 mouse, which harbors a human transgene comprising APPK^{670N/M671L} and PSEN1^{ΔE}9[30]. Male mice were used instead of female mice, and FSH Ab was injected beginning at 5 months of age. Unlike 3xTg mice, APP/PS1 show a less pronounced phenotype and develop amyloid plaques at 6 months, overt cognitive impairment at a later age of ˜12 months, and importantly, do not display neurofibrillary tangles (Minkeviciene, R. et al. J Neurochem 105, 584-594 (2008); Onos, K. D. et al. PLoS Genet 15, e1008155, (2019); Volianskis, A., et al. Neurobiol Aging 31, 1173-1187, 2008.08.005 (2010)). Therefore, the ability of the FSH Ab to prevent deposition of Aβ (escalating doses from 120 to 150 μg/mouse, i.p. 5 days/week) was tested for 4 months. Prior to sacrifice, the Novel Object Recognition test for learning and recognition memory was conducted. FSH Ab prevented Aβ40 and Aβ42 accumulation in both hippocampus and cortex (FIG. 1F). No Aβ isoforms were noted in wild type mice with or without FSH Ab (FIG. 1F). As APP/PS1 mice display overt cognitive decline at ˜12 months, no evidence was found in novel object discrimination index or its alteration with FSH Ab at 9 months of age (FIG. 6J). Of note is that ˜35% of the mice experienced sudden death from seizures, which are known to occur on a C57BL/6 background.

### Conclusion

Collectively, the data with 3xTg and APP/PS1 mice provide unequivocal evidence for rescue by FSH inhibition of the neuropathology and cognitive decline reminiscent of human AD. The neuropathology and cognitive decline in ovariectomized 3xTg mice were likely exacerbated by FSH elevations, and thus rescued by the FSH Ab. In contrast, FSH Ab prevented Aβ accumulation in male APP/PS1 mice by suppressing the action of FSH at basal circulating levels. Studying the action of FSH inhibition in male mice is critical as the ˜3.5% annual increase in serum FSH levels in men (Araujo, A. B. & Wittert, G. A. Clinical Endocrinology & Metabolism 25, 303-319, (2011)) tracks with the onset of AD. Further, and importantly, FSH Ab caused an early rescue of Aβ accumulation prior to the onset of cognitive impairment, suggesting that the FSH Ab could be used for preventing cognitive dysfunction in high risk AD in both genders.

### Example 2: FSHRs Trigger C/EBPβ-AEP/S-Secretase Activation Via AKT and ERK1/2 in Neurons

Noting the effect of the highly specific FSH Ab in reducing AD pathology, the expression of FSHRs was determined in human and rodent brains. PCR showed mRNA expression in human cortex, human neuroblastoma (SH-SY5Y) cells, mouse cortex and hippocampus, and rat neurons (FIG. 2A). Real time PCR confirmed Fshr mRNA expression in mouse hypothalamus, hippocampus and cortex, expectedly at significantly lower levels than the ovary (FIG. 2B). Western immunoblotting further showed an ˜85 kDa FSHR protein in both male and female whole mouse brains (FIG. 2C).

To study whether FSH acts on the brain FSHR to activate the C/EBPβ-AEP/δ-secretase pathway, human recombinant FSH (30 ng/ml) was used to stimulate human neuroblastoma SH-SY5Y cells. Western immunoblotting showed that FSH increased total and phosphorylated C/EBPβ and cleaved (active) AEP in a time-dependent manner and that the active AEP subsequently cleaved both APP and Tau to the truncated forms, APP¹⁻⁵⁸⁵ and Tau¹⁻³⁶⁸, respectively (FIG. 7A). Consistent with this, quantitative PCR showed time-dependent increases in mouse Cebpb, Lgmn, App and Mapt mRNA and enhanced AEP activity upon FSH exposure (FIGS. 7B and 7C). FSH also induced the expression of pro-inflammatory cytokines IL1β, IL-6 and TNFα (FIG. 2D); this is consistent with the proinflammatory responses to C/EBPβ activation (Straccia, M. et al. J Neuroinflammation 8, 156, (2011); Caivano, M., et al. J Biol Chem 276, 48693-48701, (2001)).

To establish conclusively that the effects of FSH were mediated by the FSHR, FSHR was knocked down using siRNA-Fshr (siFshr), with scrambled siRNA as control (FIG. 2E). siFshr attenuated FSH-mediated enhancements in total and phosphorylated C/EBPβ; AEP, APP and Tau cleavage; mouse Cebpb, Lgmn, App, and Mapt mRNA; AEP enzymatic activity; and secreted IL-1β, IL-6 and TNFα (FIGS. 7E-7H). To obtain further, more definitive evidence for a function of the C/EBPβ-AEP/δ-secretase pathway in FSH-induced AD pathology, rat primary cortical cultures were infected with a sh-Cebpb-containing lentivirus (LV) to knock down C/EBPβ, and an adeno-associated virus (AAV) expressing inactive AEP, namely AAV-AEP^{C189S}. Knocking down C/EBPβ or inactivating AEP dramatically reduced the FSH-induced cleavage of APP to APP¹⁻⁵⁸⁵ and Tau to Tau¹⁻³⁶⁸ (FIG. 2D). Immunofluorescence co-staining confirmed that the enhancements in Aβ, cleaved APP and Tau, and pTau in response to FSH were reduced in cells infected with LV-sh-Cebpb or AAV-AEP^{C189S} (FIG. 8A). Likewise, increased AEP activity was abrogated by knockdown of C/EBPβ or by inactive AEP (FIG. 8B).

To explore downstream consequences of FSHR stimulation, the effect of FSH on the phosphorylation of ERK1/2, SRPK2 and AKT was studied. Of note, MAPK-induced phosphorylation of residue T188 of C/EBPβ regulates its transcriptional activity (Ramji, D. P. & Foka, P. Biochem J 365, 561-575, (2002)). Likewise, AKT phosphorylates SRPK2, which, in turn, activates AEP by phosphorylating residue S266. Total C/EBPβ, pC/EBPβ, and active AEP was found to progressively increase from 5 minutes post-FSH exposure, while ERK1/2, AKT and SRPK2 phosphorylation peak at ˜30 min, with a delayed elevation in NF-κB p65 (FIG. 8C). Inhibitors of each pathway were utilized, including pertussis toxin that decouples the bone and adipose tissue FSHRs from Gα_{αi} protein (Liu, P. et al. Nature 546, 107-112, (2017); Sun, L. et al. FSH directly regulates bone mass. Cell 125, 247-260, (2006)). Pertussis toxin inhibited FSH-induced C/EBPβ and AEP activation and AKT phosphorylation (FIG. 2E). Absence of an effect of the adenylate cyclase inhibitor SQ22536 on any signaling molecule is consistent with a maximally-inhibited G_{α1} pathway coupled to FSH (FIG. 2E). Furthermore, the AKT inhibitor AKTi-1/2 attenuated the induction by FSH of C/EBPβexpression, AEP cleavage and phosphorylation, and phosphorylation of the downstream target SRPK2, but not ERK1/2 (FIG. 2E). In contrast, while the MEK1 inhibitor PD98059 blocked FSH-induced C/EBPβ, AEP and SRPK2 activation, AKT was unaffected (FIG. 2E).

### Conclusion

These data suggest collectively that FSHR signaling in neurons triggers AKT, ERK1/2 and SRPK2 phosphorylation leading to C/EBPβ-AEP/δ-secretase activation and subsequently, the proteolytic cleavage of APP and Tau (FIG. 2F).

### Example 3: Targeted Fshr Knockdown in the Hippocampus Reverses AD Pathology

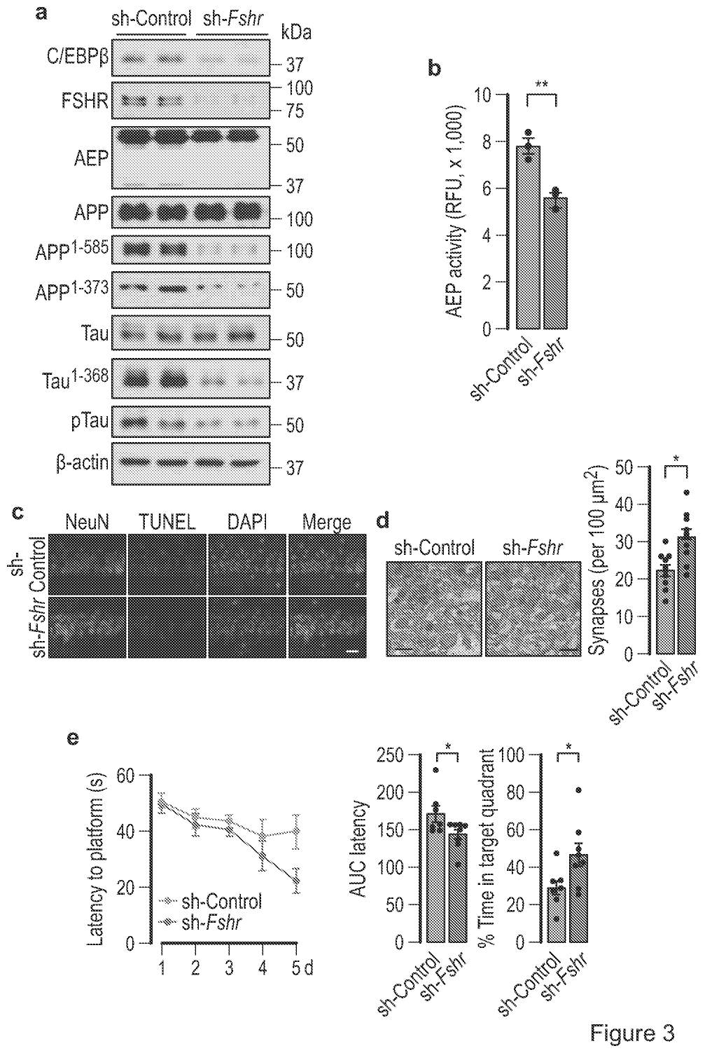

Having established the expression of a functional brain FSHR, the selective ablation of FSHR in the hippocampus in mimicking the effect of systemic administration of FSH Ab in rescuing ovariectomy-induced AD neuropathology and cognitive decline was explored. Briefly, 2 μl of AAV2-Fshr (iAAV04355302) or control (iAAV01502) viral suspension (2×10⁹ vector genomes/μl) was injected stereotactically at a rate of 0.25 μl/min into the hippocampus of 3xTg mice. 7 days after stereotaxic injection, the mice were ovariectomized, followed 3 months later, by cognitive testing and sacrifice. A significant reduction in FSHR protein and mRNA were noted in hippocampal extracts of ovariectomized mice injected with AAV2-Fshr compared with those receiving control shRNA (FIG. 3A and FIG. 9A). These AAV2-Fshr-injected mice also showed impressive decrements in total C/EBPβ and AEP, in cleaved APP, and in total, cleaved and phosphorylated Tau compared with ovariectomized mice receiving control shRNA (FIG. 3A). This inhibition was recapitulated at the mRNA level for the encoding genes, namely mouse Cebpb, Lgmn, App and Mapt genes (FIG. 9A). Likewise, compared with shRNA-injected mice, a dampening of AEP activity in AAV2-Fshr mice was accompanied by marked changes in downstream events, namely sharp reductions in Aβ40 and Aβ42 levels, and neuronal apoptosis, together with increases in dendritic spines and synapse number (FIGS. 3B-3D and FIGS. 9A-9D). Morris Water Maze testing revealed significant enhancements in learning, notably reduced latency to mount a platform and increased time spent in the platform quadrant in AAV2-Fshr-injected mice (FIG. 3E). Together, the data provide compelling evidence for a role for the hippocampal FSHR in mediating, at least in part, the effects of FSH on AD pathogenesis.

### Example 4: Systemic FSH Injection Mimics the Effect of Ovariectomy on AD Pathology

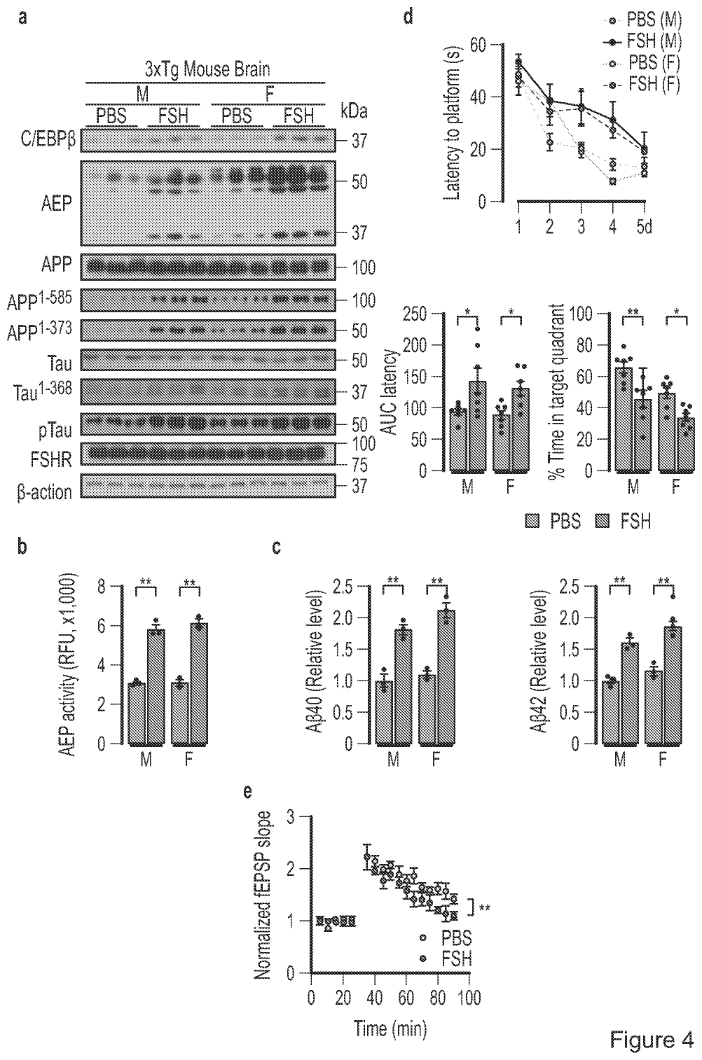

For gain-of-function studies, recombinant human FSH was injected into 2.5-month-old female 3xTg mice (5 IU/mouse) daily for 3 months. FSH injections caused a marked increase in C/EBPβ and AEP in 3xTg brains compared with PBS injections (FIG. 4A). The robust activation of AEP/δ-secretase induced cleavage of APP and Tau to produce APP¹⁻³⁷³, APP¹⁻⁵⁸⁵ and Tau¹⁻³⁶⁸, and phosphorylated Tau (FIGS. 4A-4B, and 10A-10B). Likewise, FSH induced pTau and Aβ, and both Aβ40 and Aβ42 isoforms (FIG. 4C and FIG. 11A), with plaque formation confirmed in hippocampal sections by thioflavin staining, and in the cortex and hippocampal regions, CA1 and dentate gyrus, by silver staining (FIGS. 10C and 11B).

Consistent with elevated protein levels (FIG. 4A), FSH also increased Cebpb, Lgmn, App, and Mapt mRNA expression in both sexes (FIG. 11C). Furthermore, FSH triggered marked apoptosis in hippocampal and cortical neurons, and reduced dendritic spine and synapse numbers in the hippocampus (FIGS. 10E-10D, and 11D-11E). Concordant with these neuropathologic changes, Morris Water Maze testing revealed increased latency to mount the platform and reduced time spent in the target quadrant following FSH injection (FIG. 4D). Electrophysiological analysis of long-term potentiation showed reduced fEPSP in the CA1 hippocampal region (FIG. 4E). In all, systemic FSH injection over 3 months resulted in a marked acceleration of AD pathology in 3xTg mice, in essence replicating the effect of elevated levels of endogenous FSH following ovariectomy.

Given that FSH increases serum estrogen, mice were ovariectomized and serum estrogen levels were clamped close to basal (˜75 μg/ml) by subcutaneously implanting 17β-estradiol 90-day-release-pellets (0.36 mg) before FSH (5 IU daily, i.p.) injections for 3 months (FIG. 12A). FSH injections, even in the presence of clamped estrogen levels (estrogen-replete), continued to induce C/EBPβ and AEP expression, cleavage of APP and Tau, and Tau phosphorylation (compare FIG. 4A vs FIG. 12B). Likewise, in these ovariectomized, but estrogen-replete (eugonadal) mice, FSH induced AEP/δ-secretase activation, and, as a consequence, led to Aβ and pTau accumulation (compare FIG. 4B, FIGS. 11A-11B, and FIGS. 12C-12D). This experiment provides unequivocal evidence that the effect of FSH is independent of estrogen.

### Example 5: FSH Induces AD Pathology Via C/EPBβ

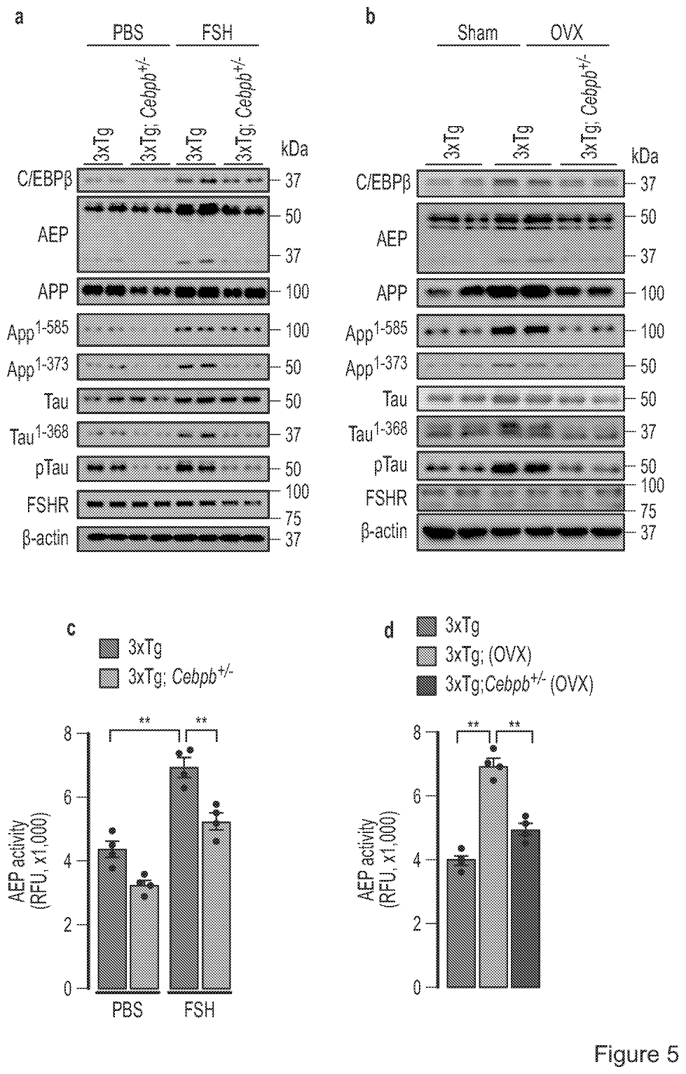

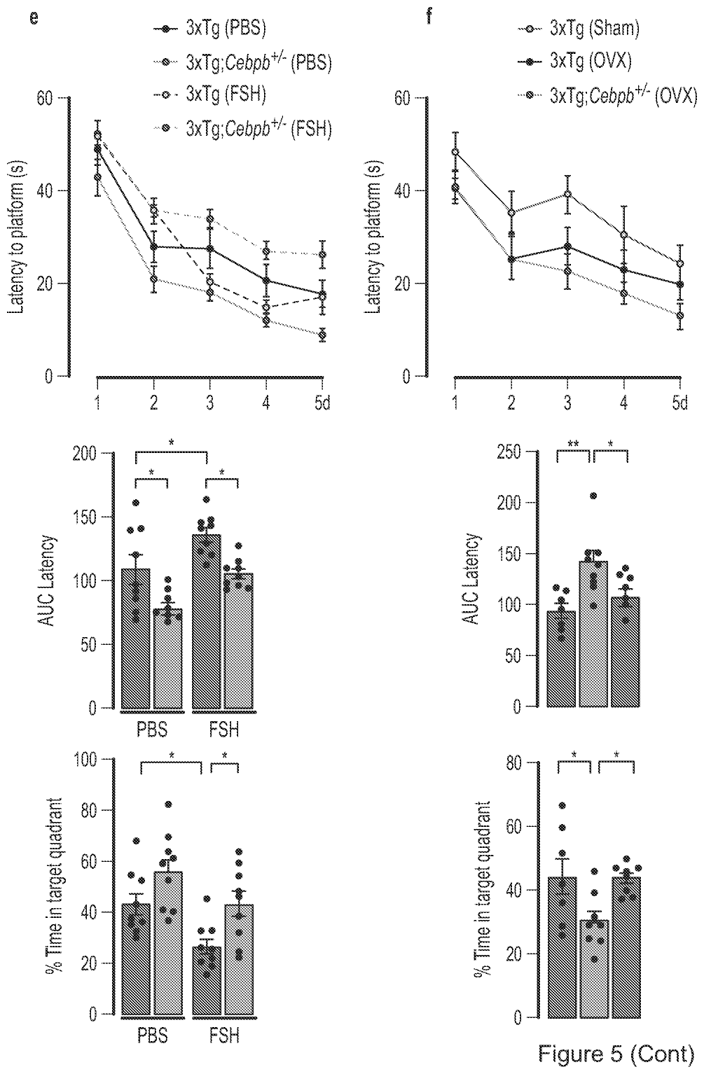

Compound 3xTg;Cebpb^{+/-} mutants were generated on a C57BL/6 background by crossing 3xTg with Cebpb^{+/-} mice. 2.5- to 3-month-old compound mutants were treated with recombinant human FSH (5 IU/mouse) daily for 3 months. Of note is that Cebpb-mice were not utilized due to a potentially confounding metabolic phenotype (Millward, C. A. et al. Diabetes 56, 161-167, (2007)). Haploinsufficiency of Cebpb resulted in attenuated downstream events at baseline—notably, AEP activation and APP and Tau cleavage were diminished (FIGS. 5A and 5C). FSH treatment of 3xTg mice markedly increased AEP/δ-secretase activity and consequently led to the accumulation of APP¹⁻³⁷³ APP¹⁻⁵⁸⁵, Tau¹⁻³⁶⁸ and pTau; all responses to FSH were attenuated in 3xTg;Cebpb^{+/-} mice (FIG. 5A). Likewise, FSH-induced Cebpb, Lgmn, App and Mapt expression was attenuated in 3xTg;Cebpb^{+/-} mice compared with 3xTg littermates (FIG. 13A). Immunohistochemistry revealed that FSH enhanced both Aβ and pTau signals in the hippocampus of 3xTg mice, which were again sharply reduced in 3xTg;Cebpb^{+/-} mice (FIGS. 13B-13D). C/EBPβ haploinsufficiency also reversed the marked decrements in dendritic spine numbers induced upon FSH treatment (FIG. 13E). Consistent with the reduction of FSH-induced AD neuropathology upon C/EBPβ depletion, Morris Water Maze testing showed a reversal of the cognitive deficits induced by FSH, namely reduced latency to mount the platform and enhanced time spent in platform quadrant (FIG. 5E).

Given these results, and the premise that elevated serum FSH levels contribute to the acceleration of AD following ovariectomy (FIG. 1), the effects of ovariectomy was examined in compound 3xTg;Cebpb^{+/-} mice. Thus, it was predicted that in compound mutants, the effects of ovariectomy on AD pathology and cognitive decline will phenocopy that of recombinant FSH injections into these mice (FIG. 4). As before (FIG. 1), ovariectomy of 3xTg mice induced C/EBPβ production; activated AEP/δ-secretase to generate APP¹⁻³⁷³, APP¹⁻⁵⁸⁵, Tau¹⁻³⁶⁸ and p-Tau; increased Cebpb, Lgmn, App and Mapt mRNA; enhanced Aβ and pTau accumulation; triggered Aβ plaque formation; and reduced dendritic spine number (FIGS. 5B and 5D and FIGS. 14A-14D). These data were consistent with cognitive testing, which revealed attenuation in 3xTg;Cebpb^{+/-} mice of FSH-induced responses, namely increased latency to mount a platform and reduced time spent in the platform quadrant (FIG. 5F). In all, these data provide strong genetic evidence for a primary role for C/EBPβ in mediating the AD pathology induced by FSH.

The Examples of this disclosure demonstrate that FSH receptors are present in hippocampal and cortical neurons; that FSH induces Aβ and Tau accumulation; that systemically administered FSH phenocopies the neuropathology and cognitive impairment noted post-ovariectomy in AD mice; that FSH action is mediated through C/EBPβ and AEP/δ-secretase activation; and importantly, that anti-FSHβ antibody rescues AD progression in ovariectomized 3xTg mice and prevents AD pathology in male APP/PS1 mice. The reversal in ovariectomized 3xTg mice is mimicked by the targeted depletion of FSHRs in hippocampal neurons, in essence, specifying the precise site of action of FSH in the brain.

Without being bound by theory, it is believed that the examples of this disclosure link FSH and AD via the C/EBPβ-AEP/δ-secretase pathway, taken together with the strong clinical association of AD with rising serum FSH levels, which provide the basis for the use of anti-FSH antibodies and/or other anti-FSH agents for the prevention and therapy of AD in women. Furthermore, as FSH levels also rise in aging men, the exemplified finding that systemic FSH induces and the anti-FSH Ab rescues AD pathology in both male and female mice enables the use of anti-FSH antibodies in both genders. Blocking FSH has been shown to reduces bone loss, body fat and serum cholesterol (Zhu, L. L. et al. Proc Natl Acad Sci USA 109, 14574-14579 (2012). Ji, Y et al. (2018). Proc Natl Acad Sci USA 115, 2192-2197; Liu, P. et al. (2017). Nature 546, 107-112; Guo, Y et al. (2019). Cell Res 29, 151-166), and as shown in the Examples above, dampens AD pathology.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims. All references cited herein are incorporated by reference in their entireties.

What is claimed is:

1. A method of treating Alzheimer's Disease (AD), preventing the onset of AD, or reducing cognitive or functional decline in AD, in a subject in need or at risk thereof, comprising administering to said subject a therapeutically effective amount of a composition comprising an FSH-inactivating agent.

2. The method of claim 1, wherein the subject is female.

3. The method of claim 1, wherein the subject is male.

4. The method of claim 2, wherein the subject is perimenopausal or postmenopausal.

5. The method of any one of claims 1-4, wherein the subject has a condition in which FSH levels are elevated.

6. The method of claim 5, wherein the condition is a genetic disease, chemotherapy, surgical menopause, or orchiectomy.

7. The method of claim 6, wherein the genetic disease is Turners syndrome.

8. The method of any one of claims 1-7, wherein the method alters one or more of the following in the subject in need thereof:
(a) reduces Aβ accumulation;
(b) reduces amyloid plaques;
(c) reduces Tau accumulation in the brain; and
(d) enhances cognitive function.

9. The method of claim 8, wherein the one or more of Aβ accumulation, amyloid plaques, and Tau accumulation in the brain is lower by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the subject or in a control.

10. The method of claim 8, wherein the cognitive function is enhanced by at least about 20%, at least about 30%, at least about 40%, or at least about 50%, at least about 60%, at least about 70%, at least about 80%, or at least about 90%, as measured on one or more tests selected from the group consisting of the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test; the Verbal Paired Associates subtest; the Logical Memory subtest: the Visual Reproduction subtest of the Wechsler Memory Scale-Revised (WMS-R); the explicit 3—alternative forced choice task; and the Benton Visual Retention Test.

11. The method of any one of claims 1-10, wherein the subject is concurrently treated with one or more agents selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, an adeno-associated virus delivery of NGF, CERE-110, a beta-blocker, a human amyloid vaccine, a beta or gamma secretase inhibitor, a nicotinic or muscarinic agonist, and a second antibody.

12. The method of claim 11, wherein the cholinesterase inhibitor is selected from the group consisting of galantamine, rivastigmine, tacrine, and donepezil.

13. The method of claim 11, wherein the NMDA receptor antagonist is selected from the group consisting of ketamine, methadone, memantine, amantadine, and dextromethorphan or a salt thereof.

14. The method of claim 11, wherein the antipsychotic agent is selected from the group consisting of aripiprazole, risperidone, olanzapine, quetiapine, or haloperidol.

15. The method of claim 11, wherein the benzodiazepine is selected from the group consisting of lorazepam, oxazepam and temazepam.

16. The method of claim 11, wherein the tricyclic antidepressant is nortriptyline.

17. The method of claim 11, wherein the hormone is selected from the group consisting of estrogen, progesterone and leuprolide.

18. The method of claim 11, wherein the vitamin selected from the group consisting of folate and nicotinamide.

19. The method of claim 11, wherein the second antibody is selected from the group consisting of bapineuzumab, solanezumab, gantenerumab, crenezumab, ponezumab, BAN2401, and aducanumab.

20. The method of any one of the preceding claims, wherein the FSH-inactivating agent is administered intravenously, intrathecally, intracranially, cutaneously, subcutaneously, intraperitoneally, intramuscularly, orally, topically, transdermally, nasally, or rectally to the subject.

21. The method of claim 1, wherein the FSH-inactivating agent comprises an anti-FSH antibody, or antigen-binding portion thereof, wherein the anti-FSH antibody, or antigen-binding portion thereof specifically binds to one or more epitopes within a β-subunit of Follicle stimulating hormone (FSH).

22. The method of claim 21, wherein the one or more epitopes within the β-subunit of FSH comprise SEQ ID NO: 1 or SEQ ID NO: 2 or a peptide sequence consisting essentially of SEQ ID NO: 1 or SEQ ID NO: 2 but having conservative substitutions.

23. The method of claim 21, wherein the anti-FSH antibody or antigen-binding portion thereof is selected from the group consisting of a monoclonal antibody, a polyclonal antibody, a chimeric antibody, a human antibody, a single-chain variable fragment (scFv), and combinations thereof.

24. The method of claim 24, wherein the anti-FSH antibody is a polyclonal antibody.
